(19) United States
(12) Patent Application Publication
(10) Pub. No.: US 2005/0118165 A1
(43) Pub. Date: Jun. 2, 2005

(54) Monoclonal antibody imaging and therapy of tumors that express met and bind hepatocyte growth factor

(75) Inventors: Rick V. Hay, Ada, MI (US); Boliang Cao, Ada, MI (US); James H. Resau, Grand Rapids, MI (US); George F. Vande Woude, Ada, MI (US); Milton D. Gross, Ann Arbor, MI (US)

Correspondence Address: VENABLE, BAETJER, HOWARD AND CIVILETTI, LLP, P.O. BOX 34385, WASHINGTON, DC, 20043-9998, US

(73) Assignee: Van Andel Institute; and the USA as Represented by the Department Of Veterans Affairs, Grand Rapids, MI (US)

(21) Appl. No.: 10/500,297

(22) PCT Filed: Dec. 27, 2002
(86) PCT No.: PCT/US02/41607

Related U.S. Application Data
(60) Provisional application No. 60/342,398, filed on Dec. 27, 2001.

(51) Int. Cl. A61K 39/395; C07K 16/44

(52) U.S. Cl. 424/141.1; 530/388.15

(57) ABSTRACT

In a wide variety of human solid tumors, an aggressive, metastatic phenotype and poor clinical prognosis are associated with expression of the receptor tyrosine kinase. Met and its agonist ligand HGF. Disclosed herein are (a) mAbs and hybridoma cell lines that produce them, which mAbs antibodies are specific for Met and (b) combinations of anti-Met and anti-HGF mAbs. When detectably labeled, these antibodies are useful for imaging such tumors. Anti-Met mAb compositions and methods for scintigraphic detection, diagnosis, prognosis, monitoring and therapy of Met-bearing tumors are provided.

### BACKGROUND OF THE INVENTION

1. Field of the Invention

The present invention in the field of medicine, immunology and cancer diagnosis and therapy, is directed to monoclonal antibody (mAb) compositions that are useful for imaging and treating tumors that express the Met oncogene product and bind hepatocyte growth factor/scatter factor.

2. Description of the Background Art

In the field of cardiovascular medicine, biomedical imaging has succeeded in visualization and quantitation of factor that permit a reasonable assessment of risk and, therefore, in guiding therapeutic choices. We routinely assess myocardial perfusion by noninvasive imaging methods in conjunction with physical or pharmacological stress testing, a process known as “cardiac risk stratification.” While the field of oncology lags behind, recent developments are leading to the development of parallel approaches that may permit most or all patients with newly diagnosed, clinically-confined cancers to undergo a test or tests that would serve as (or contribute to) a “metastatic risk stratification” (MRS). A person with a low MRS score would be considered to have a tumor at low risk of metastatic or invasive behavior, and could be monitored and treated conservatively; one with an intermediate MRS score could be treated conservatively but monitored frequently; and one at high risk by MRS would have an objective basis for agreeing to and enduring a correspondingly more aggressive therapy and intensive monitoring protocol. Through MRS, we could objectively individualize the treatment and monitoring of patients with cancer in a way that has not heretofore been thinkable.

Every dividing cell has the potential to become neoplastic, and every neoplasm has the potential to become frankly malignant, i.e., able to invade and metastasize. For over 20 years, molecular oncologists have sought molecules that are important in, characteristic of, and potentially diagnostic for, carcinogenesis and cancer progression for over twenty years. Now, armed with the technical ability to perform high-throughout gene expression microarray analysis and proteomic analysis on thousands of molecules at a time, the process is accelerating (Takahashi M et al., 2001, Proc Natl Acad Sci USA 98:9754-9759 and PCT publication WO02/07941 A2; Huang Y et al., 2001, Proc Natl Acad Sci USA 98:15044-15049; Miller J C et al., “Antibody microarray profiling of human prostate cancer sera: antibody screening and identification of potential biomarkers.” Proteomics, in press, 2002). There is an ever-growing list of candidate molecules that might help determine “very malignant” status, or that can serve as extratumoral indicators of that status, for every type of cancer that has been interrogated with this technology. It is expected that from the growing mountain of data, at least a few molecules will emerge as useful markers of and targets for treating very malignant cancers. On the other hand, several molecules whose presence and form of expression are related to metastatic risk were known before the recent explosion in gene expression analysis technology. The time is right to begin exploiting these molecules for MRS, or at least as prototypes for the MRS algorithms of the future. One such example is the molecule known as Met.

Met, the protein product of the c-met-protooncogene, was discovered and studied in the laboratory of George Vande Woude at the National Cancer Institute beginning in 1984 (Cooper C S et al., 1984, Nature 311:29-33; Dean M et al., 1985, Nature 318:385-388; Iyer A et al., 1990, Cell Growth Differ 1:87-95) Met is a receptor protein tyrosine kinase of the same family as epidermal growth factor (EGF) receptors. This transmembrane protein acts as the cell surface membrane receptor in which the extracellular domain (ECD) binds hepatocyte growth factor/scatter factor (HGF/SF, also abbreviated HGF herein). Met dimerizes after binding ligand to form the active kinase. The intracellular tyrosine kinase domain activates a complex cascade of biochemical reactions. Under normal conditions Met is a keystone molecule, acting on the molecular signaling pathways responsible for cellular differentiation, motility, proliferation, organogenesis, angiogenesis, and apoptosis (Haddad R et al., 2001, Anticancer Res 21:4243-4252). In neoplastic cells the aberrant expression of Met and HGF leads to emergence of an invasive/metastatic phenotype. Supporting this are results of transfection experiments and retrospective analyses of many types of human solid tumors, including cancers originating in the head and neck, thyroid, lung, breast, stomach, liver, pancreas, colon and rectum, kidney, urinary bladder, prostate, ovary, uterus, skin, bone, muscle, and other connective tissues [Haddad et al., supra; (Stuart, K A et al (2000) Int J Exp Path 81:17-30; van der Voort, R et al. (2000) Adv Cancer Res 79:39-90). Both paracrine and autocrine mechanisms of Met activation by HGF occur in human neoplasms. Moreover, activating mutations in Met—either inherited in the germ line or found in sporadic cancers—have been shown to contribute to a variety of human cancers (Schmidt L et al., 1997, Nat Genet 16:68-7313).

Across the spectrum of tumors, levels of Met-HGF expression in general correlate inversely with clinical outcome. This correlation has been examined in greatest detail for human breast and prostate carcinomas. Met overexpression in breast tumors is associated with breast cancer progression (Niemann C et al., 1998, J Cell Biol 143:533-545; Tsarfaty I et al., 1999, Anal Quant Cytol Histol 21:397-408; Firon M et al., 2000, Oncogene 19:2386-2397) and high HGF expression also correlates with poor survival in ductal breast carcinomas (Yamashita J I et al., 1994, Cancer Res 54:1630-1633; Ghoussoub RAD et al., Cancer 82:1513-1520). Tsarfaty et al., supra quantified Met expression in uninvolved (N) relative to tumor (T) tissue in the same primary breast carcinoma sections. The overall Met distribution in this patient group was ˜40% with T<N, ˜40% with N=T, and 20% with T>N. Higher Met expression in tumors than in normal tissue was associated with poor patient outcome.

Three groups (Jin L et al., 1997, Cancer 79:749-760; Tuck A et al., 1996, Am J Pathol 148: 225-232; Edakuni G et al., 2001, Pathol Int'l 51:172-178) have examined Met and HGF expression in benign and malignant breast tissue and found that, frequently, both receptor and ligand are expressed, and that expression is higher in breast cancer and in carcinomas in situ than in benign breast tissue. While Met is mainly detected in epithelial breast cancer cells, HGF is detected in tumor cells as well as in stromal cell types, implying that HGF contributes to growth and invasiveness of breast cancer cells by either or both autocrine and paracrine mechanisms. This conclusion is also supported by results showing increased tumorigenic and metastatic activity accompanied by reduced tubule formation of breast cancer cells after transfection with Met and HGF (Firon et al., supra). There is a growing body of clinical and experimental evidence that Met also plays a critical role in the behavior of human prostate carcinoma. Four independent laboratories have reported aberrant expression of Met by about one-half to two-thirds of localized prostate cancers, but evidently by all bone metastases of these tumors. This suggests that Met provides a strong selective mechanism for metastatic growth in prostate cancer (Humphrey P A et al., 1995, Am J Pathol 147:386-396; Pisters L L et al., 1995, J Urol 154:293-298; Watanabe M et al., 1999, Cancer Lett 141:173-178; Knudsen B S et al., 2002, Urology, 60:1113-1117)).

Put simply, until or unless something better comes along, Met can be considered the “poster child” of very malignant cancers in that (1) very malignant cancers express Met independently of the tissue of origin, and (2) Met is a process-specific rather than tissue-specific marker for cancer, an indicator of tumor destiny rather than of tumor origin.

With these notions in mind, the present inventors set out to utilize molecular imaging to exploit Met in order to determine the status of Met expression in particular solid tumors in vivo, and armed with that information, to design Met-directed therapies that will alter tumor destiny toward a more favorable clinical outcome.

The present disclosure describes the development of molecular imaging tools and approaches to clarify the behavior of Met at the cellular level, and apply these approaches to in vivo animal models of human cancer and to naturally occurring human cancers.

The present inventors and their colleagues approaches exploiting Met as a molecular imaging and therapeutic target fall into four general areas:
1. Microscopic molecular imaging: Immunohistochemistry, immunofluorescence (IF), and confocal laser scanning microscopy (CLSM)
2. Nuclear molecular imaging: Radioimmunoscintigraphy
3. “Provocative” functional molecular imaging: Assessing tumor physiology by magnetic resonance imaging and ultrasonography
4. Met-directed forms of cancer therapy.
   The present invention is primarily focused on approach #2, leading to developments under #4, above.

A number of publications disclose anti-Met antibodies. U.S. Pat. Nos. 5,686,292, 6,207,152, 6,214,344 to Schwall et al. (Nov. 11, 1997, Mar. 27, 2001, and Apr. 10, 2001, respectively disclose mAbs, particularly monovalent antibodies that are antagonists of the HGF receptor and their uses in treating cancer. None of these documents mention in vivo diagnosis using these antibodies or fragments.

U.S. Pat. No. 6,099,841 (Hillan et al.), Aug. 8, 2000, discloses antibodies and fragments that are HGF receptor agonists. The document discloses that these molecules can be employed to substantially enhance HGF receptor activation, may be included in pharmaceutical compositions, articles of manufacture, or kits. Methods of treatment and in vitro diagnosis using these molecules HGF receptor agonists are also disclosed. All that is disclosed regarding in vivo diagnosis is a vague statement that “[v]arious diagnostic assay techniques known in the art may be used, such as in vivo imaging assays . . . . ” The only in vivo use given more attention is that of stimulating hepatocyte proliferation.

Prat et al., Mol Cell Biol 11:5954-5962 (1991) described several mAbs specific for the extracellular domain of the β-chain encoded by the c-Met gene (see also, WO 92/20792). The mAbs were selected following immunization of mice with whole live GTL-16 cells (human gastric carcinoma cell line) overexpressing Met. Hybrid supernatants were screened for binding to GTL-16 cells. Four mAbs referred to as DL-21, DN-30, DN-31 and DO-24, were selected. Prat et al., Int J Canc 49:323-328 (1991) described using anti-c-Met mAb to detect distribution of the Met protein in human normal and neoplastic tissues. See, also, Yamada et al., Brain Res 637:308-312 (1994). The mAb DO-24 was reported to be an IgG2a isotype antibody.

Crepaldi et al., J Cell Biol 125:313-320 (1994) reported using mAbs DO-24 and DN-30 (supra) and mAb DQ-13 to identify subcellular distribution of HGF receptors in epithelial tissues and in MDCK cell monolayers. According to this document, DQ-13 was raised against a peptide corresponding to 19 C-terminal amino acids (from Ser¹³⁷² to Ser¹³⁹⁰) of human c-Met.

A mAb specific for the cytoplasmic domain of human c-Met was described by Bottaro et al., Science 251:801-804 (1991).

Silvagno et al., Arterioscler Thromb Vasc Biol 15:1857-1865 (1995) described using a Met agonist antibody in vivo to promote angiogenesis in Matrigel® plugs.

According to Hillan et al., supra; several of the mAbs cited above were commercially available from Upstate Biotechnology Incorporated, Lake Placid, N.Y. (DO-24 and DL-21, specific for an extracellular epitope and DQ-13 specific for an intracellular epitope).

Tumor Imaging

Radioimmunoscintigraphy is an important and attractive modality for experimental and clinical molecular imaging of cancer. One can raise, characterize, and propagate mAbs reactive against virtually any given protein antigen, even those present as minor components of complex protein mixtures or as minor surface components of whole cells. Established methods for radiolabeling mAbs in suitable quantity and of appropriate quantity for scintigraphy are available, feasible, relatively inexpensive, and adaptable to virtually any mAb regardless of its epitopic specificity. New radiolabeling methods are continually emerging, and many laboratories are evaluating a wide range of antibody derivatives—from full-length chimeric and humanized molecules, to monomeric and multimeric antibody fragments, to immunoconjugates—as potentially superior imaging and therapeutic agents, with improved targeting selectivity and more favorable biological turnover kinetics (Program and Abstracts, Ninth Conference on Cancer Therapy with Antibodies and Immunoconjugates. 2002. Cancer Biotherapy & Radiopharmaceuticals 17:465-494).

Moreover, the reagents, supplies, and equipment required to perform radioimmunoscintigraphy in experimental animals and in humans are commonplace. For decades decommissioned or refurbished clinical gamma cameras have proven satisfactory for animal imaging applications, and they continue to do so. Modified or custom-built gamma cameras adapted for small animal imaging are becoming more widely available.

The major advantage of scintigraphy as a molecular imaging modality (not limited to imaging with antibodies) is that the acquired images are inherently quantitative. The physics of gamma radiation and the mathematical analysis of nuclear images, including corrections for photon attenuation and other artifacts, are well understood. In animal models as well as in human studies we can noninvasively and accurately measure net accumulation and some kinetic parameters of radiopharmaceutical interactions with target lesions, and the concurrent collection of even a small set of biological samples (e.g., blood and excreta) for direct counting combined with quantitative analysis of diagnostic images enables us to make useful dosimetry estimates for therapeutic purposes.

Many different radiopharmaceuticals are available for imaging neoplasms. They range from classical agents such as sodium iodide (Na-¹³¹I, thallium chloride (²⁰¹TlCl), and gallium citrate (⁶⁷Ga-citrate) to highly selective positron-emitting reporter gene detection systems (Vallabhajosula S (2001), In: Nuclear Oncology. I Khalkhali et al., eds. Lippincott Williams & Wilkins, Philadelphia, Pa. pp. 31-62; Iyer M et al. (2001) J Nucl Med 42, 96-105). Radiolabeled molecules that bind to specific cell surface components provide one successful approach to tumor imaging and therapy. Examples are OctreoScan® for imaging and potentially treating neuroendocrine neoplasms, CEAScan® and OncoScint® for imaging colorectal and ovarian cancers, and Bexxar® and Zevalin® for detecting and treating certain lymphomas.

As a novel variation of that strategy, the present inventors have begun to develop radiopharmaceuticals (as well as related diagnostic and therapeutic agents) that are designed to distinguish neoplasms according to their genotype and invasive/metastatic potential rather than by tissue of origin, based on targeting of the Met oncogene product.

### SUMMARY OF THE INVENTION

As a novel variation of using tissue-specific mAbs as diagnostic and therapeutic agents, the present inventors have developed antibody-based agents, exemplified in the form of radiopharmaceuticals, that distinguish neoplasms according to their genotype and invasive and/or metastatic potential rather than by their tissue of origin. Such antibodies are specific for extracellular epitopes of the Met oncogene protein product. The present inventors raised and characterized mAbs against the ECD of human Met (“hMet” or “huMet”); they also produced antibodies specific for human HGF (“hHGF” or “huHGF”). They recently reported that a mixture of at least three anti-HGF mAbs with different epitope specificities, rather than a single mAb, was required to block the activation of Met by HGF in vivo (Cao B et al. (2001) Proc Natl Acad Sci USA 98:7443-74485; copending PCT application, Cao et al., WO 01/34650 which is hereby incorporated by reference in its entirety).

Disclosed herein is the imaging of tumors using a mixture of radiolabeled mAbs reactive against the Met and HGF, particularly with a tumor that produces both hHGF and hMet and is therefore stimulated to grow in an autocrine fashion. The present inventors have discovered that anti-hMet and anti-HGF antibodies or combinations thereof can be used to image in vivo human tumors expressing or secreting the protein for which these mAbs are specific (in nude mice).

Several novel anti-Met mAbs were produced against hMet and characterized. The hybridoma cell lines producing these mAbs were deposited in the American Type Culture Collection under Accession Number PTA-4349 and PTA-4477.

These antibodies (Met3 and Met5) bind to hMet in immunoassay such as ELISA or indirect IF against tumor cells known to express high levels of hMet, or by antibody inhibition of biological or biochemical activity, such as in a scatter assay or urokinase-stimulation assay.

Radioiodinated anti-hMet mAbs derived from one hybridoma designated 2F6 (=Met3), either radiolabeled alone or in combination with a neutralizing mixture of anti-hHGF mAbs, rapidly and effectively detected tumors autocrine for hMet and hHGF as demonstrated by gamma camera scintigraphy of mice bearing such tumors.

At least two anti-hMet mAbs were shown to be agonists when binding Met. At least one anti-hMet mAb was a potent antagonist when binding Met.

The present invention is thus directed to the following new mAbs
(a) a mAb Met3 produced by the hybridoma cell line deposited in the American Type Culture Collection under Accession Number PTA-4349; and
(b) a mAb Met5 produced by the hybridoma cell line deposited in the American Type Culture Collection under Accession Number PTA-4477,
   or an antigen binding fragment or derivative of the antibody.

Also intended is aha mAb, or antigen-binding fragment or derivative thereof, that has all the identifying biological characteristics of the above mAbs, fragments or derivatives.

One embodiment includes a humanized mAb (or an antigen binding fragment or derivative )specific for Met, wherein the heavy chain and/or light chain V region of the anti-Met mAb, or an antigen binding site of the V region, has all the identifying biological or structural characteristics of the corresponding regions or sites of the above new mAbs, and substantially all the remainder of the humanized mAb is of human origin. Also included is a human mAb specific for Met that binds to the same epitope as the epitope to which the above mAb (Met3 or Met5 binds, or an antigen binding fragment or derivative of the human antibody.

Also intended is a composition comprising the above mAb, fragment or derivative. This composition may further comprise one or more additional antibodies specific for a Met epitope, or may comprise an antigen-binding fragment or derivative of the additional one or more antibodies. The above composition may further comprise one or more antibodies, fragments or derivatives specific for HGF. Preferably, the anti-HGF is selected from the group consisting of:
(a) a mAb produced by the hybridoma cell line deposited in the American Type Culture Collection under Accession Number PTA-3414;
(b) a mAb produced by the hybridoma cell line deposited in the American Type Culture Collection under Accession Number PTA-3416;
(c) a mAb produced by the hybridoma cell line deposited in the American Type Culture Collection under Accession Number PTA-3413; and
(d) a mAb produced by the hybridoma cell line deposited in the American Type Culture Collection under Accession Number PTA-3412.

A preferred composition, above is diagnostically useful in that at least one of the antibodies in the composition carries (is bound to, conjugated to or labeled with) a suitable diagnostic or detectable label, preferably one detectable in vivo. Preferred detectable labels include radionuclides, PET-imageable agents, MRI-imageable agents, fluorescers, fluorogens, a chromophore, a chromogen, a phosphorescer, a chemiluminescer or a bioluminescer. Such a label permits detection or quantitation of the Met or HGF level in a tissue sample and can be used, therefore, as a diagnostic and a prognostic tool in a disease where expression or enhanced expression of Met (or its binding of HGF) plays a pathological or serves as a diagnostic marker and/or therapeutic target, particularly, cancer. A preferred radionuclide is selected from the group consisting of ³H, ¹⁴C, ³⁵S, ⁹⁹Tc, ¹²³I, ¹²⁵I, ¹³¹I, ¹¹¹In, ⁹⁷Ru, ⁶⁷Ga, ⁶⁸Ga, ⁷²As, ⁸⁹Zr and ²⁰¹Tl. A most preferred label is ¹²⁵I. Preferred in vivo detection is by radioimmunoscintigraphy.

In a diagnostic antibody composition, the fluorescer or fluorogen is preferably fluorescein, rhodamine, dansyl, phycoerythrin, phycocyanin, allophycocyanin, o-phthaldehyde, fluorescamine, a fluorescein derivative, Oregon Green, Rhodamine Green, Rhodol Green or Texas Red.

Preferably, a diagnostic label is bound to the antibody protein through one or more diethylenetriaminepentaacetic acid (DTPA) residues that are coupled to the protein. In a preferred embodiment the label is bound through one DTPA residue. Preferred diagnostic compositions for MRI wherein the antibody or antibodies are coupled to one (or more) DTPA residues, to which are bound metal atoms. One preferred diagnostic method is MRI using these labeled proteins. A number of metals (not radioisotopes) useful for MRI include gadolinium, manganese, copper, iron, gold and europium. Gadolinium is most preferred. Generally, the amount of labeled antibody needed for detectability in diagnostic use will vary depending on considerations such as age, condition, sex, and extent of disease in the patient, contraindications, if any, and other variables, and is to be adjusted by the individual physician or diagnostician. Dosage can vary from 0.01 mg/kg to 100 mg/kg of each single antibody or combination of antibodies.

The present invention provides a method for detecting the presence of Met (i) on the surface of a cell, (ii) in a tissue, (iii) in an organ or (iv) in a biological sample, which cell, tissue, organ or sample is suspected of expressing Met, comprising the steps of:
(a) contacting the cell, tissue, organ or sample with a diagnostic composition as above;
(b) detecting the presence of the label associated with the cell, tissue, organ or sample.

In this method, the contacting and the detecting may be in vitro; the contacting may be is in vivo and the detecting in vitro, or, preferably, the contacting and the detecting are in vivo. The method may be carried out for purposes of diagnosis, prognosis, and/or monitoring (e.g., post-therapy). In vivo detection is preferably of a radionuclide as above, preferably by radioimmunoscintigraphy. The method may also utilize a detectable label that is an MRI-imageable agent and use MRI to detect the binding and the localization of the Met-expressing tumor.

A method of determining the progression of Met-expressing cancer comprises:
a) contacting a tissue sample from a patient having cancer with the antibody composition as above;
b) detecting the binding of the antibodies to Met;
c) measuring the amount of Met (or HGF) in the sample; and
d) correlating the antibody binding with a clinically defined stage of cancer development.

A method for detecting the presence of Met-expressing cancer in a patient comprises:
a) contacting a tissue sample from the subject with the above antibody composition;
b) detecting the binding of the antibodies with Met (and, optionally, with HGF) in the sample, whereby increased binding of antigen to the antibodies relative to the binding of antigen from a control tissue sample to the antibodies indicates an increased amount of Met in the sample, whereby the increased amount of Met indicates the presence of cancerous tissue in the sample.

Also provided is a therapeutic composition useful for treating a Met-expressing tumor, in which at least one of the antibodies (or fragment or derivative) carries a suitable therapeutic “label” also referred to herein as a “therapeutic moiety.” A therapeutic moiety is an atom, a molecule, a compound or any chemical component added to the protein that renders it active in treating a disease or condition associated with expression of Met and HGF. The therapeutically active moiety may be bound directly or indirectly to the protein. The therapeutically labeled polypeptide (antibody, fragment, derivative) protein is administered as pharmaceutical composition which comprises a pharmaceutically acceptable carrier or excipient, and is preferably in a form suitable for injection.

Preferred therapeutic moieties are radionuclides, for example ⁴⁷Sc, ⁶⁷Cu, ⁹⁰Y, ¹⁰⁹Pd, ¹²⁵I, ¹³¹I, ¹⁸⁶Re, ¹⁸⁸Re, ¹⁹⁹Au, ²¹¹At, ²¹²Pb or ²¹⁷Bi.

This invention includes an article of manufacture and a related kit. The kit may comprise
(a) a labeled first container comprising the antibody, fragment or derivative as above;
(b) a labeled second container comprising a diagnostically or pharmaceutically-acceptable carrier or excipient; and
(c) instructions for using the antibody to diagnose, prognose, monitor or treat a cancerous condition or a tumor in a subject wherein cancer or tumor cells in the subject express Met,
   wherein the antibody, fragment or derivative is effective for diagnosing, prognosing, monitoring or treating the condition and the label on the labeled container indicates that the antibody can be used for the diagnosing, prognosing, monitoring or treating, as the case may be.

Also provided is a method for inhibiting (i) the proliferation, migration, or invasion of, Met-expressing tumor cells or (ii) angiogenesis induced by Met-expressing tumor cells, comprising contacting the cells with an effective amount of the above therapeutic composition. Preferably, the contacting is in vivo.

In the treatment method, the therapeutic composition, is preferably one in which at least one of the antibodies, fragments or derivatives is bound to, conjugated to, or labeled with a therapeutic moiety.

This invention is directed to a method for treating a subject having a cancerous disease or condition associated with (i) undesired proliferation, migration or invasion of Met-expressing cells or (ii) undesired angiogenesis induced by Met-expressing cells, comprising administering to the subject an effective amount of the above therapeutic composition, preferably one in which at least one of the antibodies, fragments or derivatives is bound to, conjugated to, or labeled with a therapeutic moiety.

### BRIEF DESCRIPTION OF THE DRAWINGS

FIGS. 1A-1D shows an immunofluorescence (IF) analysis of tumors using anti-hMet mAbs. S-114 cells fixed in acetone/methanol were labeled with either (A) anti-Met mAb 2F6 followed by FITC-conjugated anti-mouse IgG (green, FIG. 1A) or (B) a polyclonal anti-Met rabbit antibody C-28 (Santa Cruz) followed by rhodamine-conjugated anti-rabbit IgG (red, FIG. 1B). FIG. 1C confirms colocalization (yellow) of the antigens recognized by the mAb and the polyclonal antibody. FIG. 1D shows a Nomarski-Differential Interference Contrast image of the cells from FIGS. 1-1C.

FIG. 2 shows a series of total body images of tumor-bearing mice injected with an ¹²⁵I-labeled mAb mixture that includes antibodies specific for hHGF and those specific for hMet. Each row of images contains serial total body scintigrams for a single tumor-bearing mouse injected with this ¹²⁵I-mAb mixture. The tumor in each mouse is indicated to the left of its row. Below each column is shown the time after mAb injection at which each image was acquired. Images were obtained in posterior projection for the upper three rows, and in anterior projection for the mouse bearing DA3. The large arrows mark the transverse positions of respective tumors. Asterisks indicate the transverse positions of thyroids. The small arrow over the 1-day postinjection image for the mouse bearing DA3 indicates urinary bladder activity. Extracorporeal radioactivity in the upper right corner of each scintigram for the mouse bearing M-114 represents a positional marker

FIGS. 3A and 3B show an ROI comparison of tumors expressing hHGF and hMet vs. tumors expressing mHGF and/or mMet. Four mice bearing tumors that grow in autocrine fashion due to hMet and hHGF (3 mice bear S-114, 1 mouse bears SK-LMS-1) and three mice bearing tumors expressing mHGF and/or m (2 mice bear DA3, 1 mouse bears M-114) were injected with an ¹²⁵I-labeled mAb mixture specific for hMet and hHGF/SF. Tumor radioactivity (T) and whole body radioactivity (WB) were quantified by “region-of-interest” analysis of serial scintigrams obtained as early as one hour and as late as 5 days postinjection, (see FIG. 2). Mean values (±1 SD) are plotted for ratios of Tt:T1h (=ratio of T at time t to T at 1 hour postinjection), WBt:WB1h, Tt:WB1h, and Tt:WBt. Differences between human and murine tumors in these mice were significant for WBt:WB1h (p≦0.001 after 1 hour) and for Tt:WBt (p<0.02 at 1 hour; p≦0.001 after 1 hour).

FIG. 4 is a schematic representation of mechanisms by which the radiolabeled mAbs bind to tumor cells. Radiolabeled anti-Met mAb (*anti-Met) is depicted as binding directly to Met expressed on the tumor cell surface. Radiolabeled anti-HGF mAb (*anti-HGF/SF) could either bind to free HGF concentrated in the extracellular milieu, thereby surrounding tumor cells with radiolabeled soluble complexes, or could form a ternary complex of mAb:HGF:Met at the cell surface.

FIGS. 5A, 5B and -5C/1-5C/3 characterize the reactivity of anti-Met mAb “Met3.” FIG. 5A shows ex vivo immunohistochemical staining with Met3. A formalin-fixed, paraffin-embedded sample of human prostate tissue was examined immunohistochemically with Met3. Met expression is shown by dark brown staining in normal prostate epithelium. The staining is most prominent in the basal cell layer (arrow). FIG. 5B shows that Met3 binds Met in cultured normal human prostate epithelial cells. A primary culture of normal human prostate epithelial cells was examined by IF with Met3 (green; left half of FIG. 5B) and with C-28 polyclonal antibody (red; right half of FIG. 5B). Antibody binding co-localizes in the plasma membrane. FIG. 5C/1-5C/3 shows that Met3 binds to the surfaces of PC-3 and DU145 prostate cancer cells: FACS analysis with Met3 (thicker green curve shifted to the right) shows surface staining in the Met-expressing PC-3 and DU145 cell lines, but not in the LNCaP cell line (which exhibits very low levels of Met expression).

FIG. 6 shows Met expression by selected human cancer cell lines. The indicated cultured cell lines were grown in DMEM containing 10% fetal bovine serum (FBS) to near-confluency. Normalized aliquots of cell lysates were subjected to SDS-polyacrylamide gel electrophoresis, electrotransfer, and immunodecoration with C-28 anti-Met polyclonal antibody (upper panel) followed by H-235 anti-β-tubulin polyclonal antibody (lower panel). Immune complexes were identified by enhanced chemiluminescence. Relevant regions of the resulting luminograms are shown.

FIG. 7 shows scintigrams of tumor xenografts. The indicated cell lines were injected subcutaneously in the posterior aspect of the right thigh or in the adjacent portion of the right flank (for melanomas) of female athymic nude mice to induce xenografts. Host animals underwent radioimmunoscintigraphy with ¹²⁵I-Met3 (50-100 μCi given intravenously when their tumors reached ≧0.5 cm in greatest dimension. A composite of serial posterior whole body scintigrams for individual animals bearing tumors as indicated on the left is shown, from 1-2 hours to 5-6 days postinjection. Arrows indicate the locations of tumor xenografts. The midline focus of activity evident near the xenograft at some time points in some animals represents radioiodide in the urinary bladder. The craniadmost focus of activity in each image represents liberated radioiodide uptake by the thyroid.

FIGS. 8A-8B show a region-of-interest (ROI) analysis of scintigrams. Serial scintigrams for each host animal were evaluated by quantitative ROI analysis. FIG. 8A depicts the estimated percent of injected activity associated with the tumor xenografts as a function of time postinjection. FIG. 8B depicts the ratio of tumor-associated radioactivity to measured total body activity as a function of time postinjection. Mean values (+1 s.d.) are shown at each time point postinjection for each xenograft group; n=3-5 animals per group.

FIGS. 9A and 9B shows the presence of activated Met in dog cells. Cells of the canine kidney cell line MDCK were cultured and exposed to HGF at the indicated concentrations. Cell lysates were prepared and immunoprecipitated with Met5 followed by electrophoresis, electrotransfer, and immunodecoration with anti-PY 4G10 (anti-phosphotyrosine antibody) to detect activated (phosphorylated) Met. SKLMS-1 cells were similarly processed as a known positive control (Met-positive, HGF-responsive).

FIG. 10, similar to FIGS. 9A/9B, shows activated Met in dog cells. Cultured MDCK cells (a canine kidney line) were exposed to HGF at the indicated concentrations. Cell lysates were immunoprecipitated with Met5 followed by electrophoresis, electrotransfer, and immunodecoration with anti-phosphotyrosine antibody to detect activated (phosphorylated) Met. SKLMS-1 cells again served as a control.

FIGS. 11A-11C show a FACS analysis of Met3 binding to PC-3 human prostate carcinoma cells. A shift of fluorescent indicator (dye-conjugated anti-mouse Ab) in the presence of Met3 to larger particle size reflects association with cells.

FIGS. 12A-12C show a FACS analysis of Met5 binding to MDCK canine kidney cells. Met5 induced a shift of fluorescent indicator (dye-conjugated anti-mouse antibody) to larger particle size reflecting association with cells.

FIGS. 13A-13D show results of nuclear imaging of human tumor xenografts with ¹²⁵I-Met5. Xenografts of the human nasopharyngeal carcinoma cell line CNE-2 and the renal cell carcinoma cell line, 769-P were grown subcutaneously in the right thighs of nude mice (3 mice/group). Each mouse was injected i.v. with ¹²⁵I-Met5, and serial gamma camera images were obtained (1 hour to 5 days postinjection). Arrows appended to the image of one mouse in each group indicate the subcutaneous (thigh) tumor locations.

### DESCRIPTION OF THE PREFERRED EMBODIMENTS

Inappropriate expression of Met and/or of its ligand, HGF correlates with poor prognosis in a variety of human solid tumors. The present inventors have developed animal models for nuclear imaging of Met and HGF expression in tumors in vivo using several novel anti-Met mAbs and/or a combination of an anti-Met mAb with one or more anti-HGF mAbs. The present inventors disclosed that Met-expressing tumor xenografts in nude mice can be visualized as early as one hour following injection of radiolabeled anti-Met alone or in combination with anti-HGF mAbs, with peak image contrast (activity in tumor vs. whole body) occurring at about three days postinjection in one case. Met-expressing tumor xenografts exhibit a range of initial uptake of the radiolabeled mAb from about 5% to 20% of the estimated injected activity. Tumor-associated radioactivity constituted from about 10 to about 40% of total body activity at peak image contrast. The turnover of radiolabeled mAbs appeared to be substantially more rapid in tumor xenografts exhibiting higher initial uptake values.

In the following description, reference will be made to various methodologies known to those of skill in the art of immunology, cell biology, and molecular biology. Publications and other materials setting forth such known methodologies to which reference is made are incorporated herein by reference in their entireties as though set forth in full. Standard reference works setting forth the general principles of immunology include A. K. Abbas et al., Cellular and Molecular Immunology (Fourth Ed.), W.B. Saunders Co., Philadelphia, 2000; C. A. Janeway et al., Immunobiology. The Immune System in Health and Disease, Fourth ed., Garland Publishing Co., New York, 1999; Roitt, I. et al., Immunology, (current ed.) C.V. Mosby Co., St. Louis, Mo. (1999); Klein, J., Immunology, Blackwell Scientific Publications, Inc., Cambridge, Mass., (1990).

Antibodies are polypeptides known also as immunoglobulin (Ig) molecules, which exhibit binding specificity to a specific antigen or epitope. The present use of the term “antibody” is broad, extending beyond the conventional intact 4-chain Ig molecule (characteristic of IgG, IgA and IgE antibodies). An antibody may occur in the form of polyclonal antibodies (e.g., fractionated or unfractionated immune serum) or a mAb (see below). Also included are Ig molecules with more than one antigen-specificity (e.g. a bispecific antibody formed by joining antigen-binding regions or chains from two different antibodies). Antibodies are typically polypeptides which exhibit binding specificity to a specific antigen. A native Ig molecule is typically a heterotetrameric glycoprotein, composed of two identical light (L) chains and two identical heavy (H) chains, with each L chain linked to a H chain by one interchain disulfide bond. Additional disulfide linkages bridge the two H chains. Each H and L chain has regularly spaced intrachain disulfide bonds. The N-terminus of each H chain and each L chain includes a variable (V) domain or region (V_{H} and V_{L}). To the C-terminal side of the V_{H} domains are a number of constant (C) domains (C_{H}); L chains have only a single C domain at its c-terminus (termed C_{L}). Particular amino acid residues form an interface between the VH and VL domains. Vertebrate L chains are assigned to one of two distinct types, also called isotypes, κ and λ, based on the amino acid sequences of their C domains. Depending on the sequence of their CH domains, Igs are members of different classes: IgG, IgM, IgA, IgE and IgD, identified by their H chains referred to respectively as γ, μ, α, ε and δ. Several subclasses or isotypes are also known, e.g., the IgG isotypes IgG₁, IgG₂, IgG₃, and IgG₄ (comprising the H chains known as γ1, ι2, γ3 and γ4, respectively), or the IgA isotypes IgA₁ and IgA₂ (comprising the H chains al and α2, respectively).

When used to described domains or regions of antibody molecules, the term “variable” refers to amino acid sequences which differ among different antibodies and which are responsible for the antibody's antigen-specificity. Sequence the variability is evenly distributed throughout the V region but is typically greater in three particular regions, termed complementarity determining regions (CDRs) or hypervariable regions, that are present in VH and VL domains. The more highly conserved portions of V domains are called the framework (FR) regions. Each VH and VL domain typically comprises four FR regions. largely adopting a β-sheet configuration, bonded to three CDRs, which form loops connecting, and in some cases forming part of, the β-sheet structure. The CDRs in each chain are held in close proximity by the FR regions and, with the CDRs from the other chain, contribute to the formation of the antigen binding site (Kabat, E. A. et al., Sequences of Proteins of Immunological Interest, National Institutes of Health, Bethesda, Md. (1987)). The C domains are not involved directly in antigen binding but exhibit various effector functions, such as opsonization, complement fixation and antibody-dependent cellular toxicity.

Also included in the definition of an antibody is an antigen-binding fragment of an Ig molecule, including, Fab, Fab′, F(ab′)₂, Fv or scFv fragments, all well-known in the art. Fab and F(ab′)₂ fragments lack the Fc fragment of intact antibody, clear more rapidly from the circulation, and may have less non-specific tissue binding than an intact antibody (Wahl et al., J. Nucl. Med. 24:316-325 (1983)). Fab fragments (and other forms of monovalent antibodies that have only a single antigen-binding site, have other known advantages, especially if it is preferred to avoid or limit internalization of the antibody into Met-bearing cells ill vivo or activation of Met and the ensuing signal transduction pathways. It will be appreciated that Fab, F(ab′)₂, Fv and scFv fragments or forms of the antibodies useful in the present invention may be used for the detection, quantitation or isolation of Met proteins and the diagnosis or therapy of Met-expressing tumors in the same manner as an intact antibody. Conventional fragments are typically produced by proteolytic cleavage, using enzymes such as papain (for Fab fragments) or pepsin (for F(ab′)₂ fragments). Fv fragments are described in (Hochman, J. et al.,1973, Biochemistry 12:1130-1135; Sharon, J, et al., 1976, Biochemistry 15:1591-1594). scFv polypeptides include the hypervariable regions from the Ig of interest and recreate the antigen binding site of the native Ig while being a fraction of the size of the intact Ig (Skerra, A. et al. (1988) Science, 240: 1038-1041; Pluckthun, A. et al. (1989) Methods Enzymol. 178: 497-515; Winter, G. et al. (1991) Nature, 349: 293-299); Bird et al., (1988) Science 242:423; Huston et al. (1988) Proc. Natl. Acad. Sci. USA 85:5879; U.S. Pat. Nos. 4,704,692, 4,853,871, 4,94,6778, 5,260,203, 5,455,030. Also included as antibodies are diabodies and multispecific antibodies formed by combining more than one antigen-binding antibody fragment from antibodies of different specificity.

A “monoclonal antibody or mAb” as used herein refers to an antibody that is part of a substantially, if not totally, homogeneous population of antibodies that are a product of a single B lymphocyte clone. mAbs are well known in the art and are made using conventional methods; see for example, Kohler and Milstein, Nature 256:495-497 (1975); U.S. Pat. No. 4,376,110; Harlow, E. et al., Antibodies: A Laboratory Manual, Cold Spring Harbor Laboratory Press, Cold Spring Harbor, N.Y., 1988); Monoclonal Antibodies and Hybridomas: A New Dimension in Biological Analyses, Plenum Press, New York, N.Y. (1980); H. Zola et al., in Monoclonal Hybridoma Antibodies: Techniques and Applications, CRC Press, 1982). mAbs maybe produced recombinantly as well, e.g., according to U.S. Pat. No. 4.816,567. mAbs maybe derived from a single species, e.g., a murine mAb or a human mAb, or may be chimeric.

The mAbs of the present invention are intended to include “chimeric” antibodies. A chimeric antibody is an Ig molecule wherein different parts of the molecule are derived from different animal species. An example is an Ig having a variable region derived from a murine mAb and a human Ig constant region. Also intended are antigen-binding fragments such chimeric antibodies. Chimeric antibodies and methods for their production are known in the art. See, for example, Cabilly et al, Proc. Natl. Acad. Sci. USA 81:3273-3277 (1984); Cabilly et al., U.S. Pat. No. 4,816,567 (Mar. 28, 1989) and U.S. Pat. No. 6,331,415 (Dec. 18, 2001); Morrison et al., Proc. Natl. Acad. Sci. USA 81:6851-6855 (1984); Boulianne et al., Nature 312:643-646 (1984); Neuberger et al., Nature 314:268-270 (1985); Sahagan et al., J. Immunol. 137:1066-1074 (1986); Liu et al., Proc. Natl. Acad. Sci. USA 84:3439-3443 (1987); Better et al., Science 240:1041-1043 (1988)). These references are hereby incorporated by reference.

Preferred chimeric antibodies are “humanized” antibodies. Methods for humanizing non-human antibodies are well known in the art. Humanized forms of non-human (e.g., murine) antibodies are chimeric Igs, chains or fragments thereof (such as Fv, Fab, Fab′, etc.,) which include minimal sequence derived from the non-human Ig. In a preferred humanized antibody, a human Ig recipient antibody receives residues from a CDR non-human species (donor or import antibody, e.g., mouse, rat, rabbit) replacing the recipient CDR with the donor CDR residues. In some instances, Fv framework residues of the human Ig may be replaced by corresponding non-human residues. Humanized antibodies may also comprise residues which are found neither in the recipient antibody nor in the imported CDR or framework sequences. In general. the humanized antibody will comprise substantially all of at least one, and typically two, V domains, in which all or substantially all of the CDR regions correspond to those of a non-human Ig and all or substantially all of the FR regions are those of the human Ig consensus sequence. The humanized antibody optimally also will comprise at least part of a human Ig C region (e.g., Fc). See, Jones et al., Nature 321:522-525 (1986); Reichmann et al., Nature 332:323-327 (1988); Presta, Curr. Op. Struct. Biol, 2:593-596 (1992); Verhoeyen et al., Science, 239:1534-1536 (1988)); U.S. Pat. No. 4,816,567)

The choice of human V domains, (V_{H} and V_{L}) to be used in making the humanized antibodies is important for reducing the antigenicity of the product when administered repeatedly to a human. According to the “best-fit” method, the sequence of the V domain of a rodent antibody is screened against the entire library of known human Variable domain sequences. The human sequence which is closest to that of the rodent is then accepted as the human FR for the humanized antibody (Sims et al., J. Immunol. 151:2296 (1993); Chothia et al., J Mol. Biol. 196:901 (1987)]. Another method uses a particular FR derived from the consensus sequence of all human antibodies of a particular subgroup of L or H chains. The same FR may be used for several different humanized antibodies (Carter et al., Proc. Natl. Acad. Sci. USA 89:4285 (1992); Presta et al., J. Immunol. 151:2623-2632 (1993)).

It is important that humanized antibodies retain their (preferably high) binding affinity for the antigen and other favorable biological properties. To achieve this, humanized antibodies are designed by a process of analysis of the parental sequences and various conceptual humanized products using three dimensional (3D) models of the parental and humanized sequences. 3D Ig models are commonly available and are known to those skilled in the art. Available computer programs illustrate and display probable 3D conformational structures of selected candidate Ig sequences. Inspection of these displays permits analysis of the likely role of certain amino acid residues in the functional capacity of the candidate Ig sequence. In this way, FR residues can be selected and combined from the consensus and import sequence so that the desired antibody characteristic is achieved. In general, the CDR residues are directly and most substantially involved in influencing antigen binding (e.g., WO 94/04679).

For production of human antibodies, transgenic animals (e.g., mice) that are capable, upon immunization, of producing a full repertoire of human antibodies in the absence of endogenous Ig production can be employed. For example, the homozygous deletion of the antibody H chain joining region (J_{H}) gene in chimeric and germ-line mutant mice results in complete inhibition of endogenous antibody production. Transfer of the human germ-line Ig gene array into such germ-line mutant mice will result in the production of human antibodies upon antigen challenge (Jakobovits et al., Proc. Natl. Acad. Sci. USA 90:2551-255 (1993); Jakobovits et al. Nature, 362:255-258 (1993); Bruggermann et al., Year in Immunol. 7:33 (1993)).

Human antibodies can also be produced in phage display libraries (Hoogenboom et al., J. Mol. Biol. 222:381 (1991); Marks et al., J. Mol. Bio., 222:581 (1991)). The techniques of Cote et al. and Boerner et al. are also available for the preparation of human mAbs (Cole et al., Monoclonal Antibodies and Cancer Therapy, Alan R. Liss, p. 77 (1985) and Boerner et al., J. Immunol, 147:86-95 (1991).

Other types of chimeric molecules or fusion polypeptides involving the present mAb or antigen-binding fragments of domains thereof, include those designed for an extended in vivo half-life. This may include first identifying the sequence and conformation of a “salvage receptor” binding epitope of an Fc region of an IgG molecule. A “salvage receptor binding epitope” refers here to an epitope or fragment of the Fc region of an IgG molecule of any isotype contributes to increasing the in vivo half-life of the particular IgG molecule (when compared to other Ig classes). Once this epitope is identified, the sequence of the mAb is modified to include the sequence and conformation of the identified binding epitope. After the sequence is mutated, the chimera is tested for longer in vivo half-life compared to the unmodfied Ig molecule or chain. If a longer half-life is not evident, the sequence is altered further to include the sequence and conformation of the identified binding epitope. Care is taken that the antigen-binding activity or other desired biological activity of this chimeric molecule is maintained. The salvage receptor binding epitope generally constitutes a region corresponding to all or part of one or two loops of a Fc domain; preferably this sequence is “grafted” in an analogous position in the anti-Met antibody fragment. Preferably, three or more residues from one or two loops of the Fc domain are transferred; more preferably, the epitope is taken from the IgG CH₂ domain and transferred to one or more of the CH₁, CH₃, or V_{H} region of the anti-Met antibody. Alternatively, the epitope from the CH₂ domain is transferred to the C_{L} or the V_{L} domain of the anti-Met antibody fragment.

Another chimeric molecule intended herein comprises the anti-Met antibody chain or fragment fused to an Ig constant domain or to an unrelated (heterologous) polypeptide such as albumin. Such chimeras can be designed as monomers, homomultimers or heteromultimers, with heterodimers preferred.

In another embodiment, the chimera comprises a anti-Met antibody fragment fused to albumin. Such chimeras may be constructed by inserting the entire coding region of albumin into a plasmid expression vector. The DNA encoding the antibody chain or fragment can be inserted 5′ to the albumin coding sequence, along with an insert that encodes a linker, e.g., Gly₄ (Lu et al., FEBS Lett 356:56-59 (1994)). The chimera can be expressed in desired mammalian cells or yeast.

In general, these various chimeric molecules can be constructed in a fashion similar to more conventional chimeric antibodies in which a Variable domain from one antibody is substituted for the V domain of another antibody. For further details n preparing such antibody-nonantibody fusions, see, for example, Capon et al., Nature 337:525 (1989); Byrn et al., Nature, 344:667 (1990)

Diabodies are small antibody fragments with two antigen binding sites, which fragments comprise V_{H} domain bonded to a V_{L} domain in the same polypeptide chain (V_{H}-V_{L}). By using a linker that is too short to allow pairing between the two domains on the same chain, the domains are forced to pair with the complementary domains of another chain and create two antigen binding sites. Diabodies are described in further detail, for example, in EP404,097; WO 93/11161; and Hollinger et al., Proc. Natl. Acad. Sci, 90:6444-6448 (1993).

An anti-idiotypic (anti-Id) antibody is an antibody which recognizes unique determinants generally associated with the antigen-binding site of another antibody. An anti-Id antibody can be prepared by immunizing an animal of the same species and genetic type (e.g., mouse strain) as the source of the mAb with the mAb to which an anti-Id is being prepared. The immunized animal will recognize and respond to the idiotypic epitopes of the immunizing antibody by producing an antibody to these idiotypic determinants (the anti-Id antibody). The anti-Id antibody may also be used as an “immunogen” to induce an immune response in yet another animal, producing a so-called anti-anti-Id antibody. The anti-anti-Id may be epitopically identical to the original mAb which induced the anti-Id. Thus, by using antibodies to the idiotypic determinants of a mAb, it is possible to identify other clones expressing antibodies of identical specificity. Anti-Id mAbs thus have their own idiotypic epitopes, or “idiotopes” structurally similar to the epitope if interest, such as a Met epitope.

Antibody Functional Derivatives and Chemically Modified Antibodies

Chemical, including, covalent modifications of anti-Met antibodies are within the scope of this invention. One type of modification is introduced into the molecule by reacting targeted amino acid residues with an organic derivatizing agent that is capable of reacting with selected side chains or the N— or C-terminal residues.

Derivatization with bifunctional agents is useful for crosslinking the antibody (or fragment or derivative) to a water-insoluble support matrix or surface for use in a purification method (described below). Commonly used crosslinking agents include, e.g., 1,1-bis(diazo-acetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate create photoactivatable intermediates that can crosslink when irradiated with light. Reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are used in protein immobilization.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the a-amino groups of lysine, arginine, and histidine side chain (see, for example, T. E. Creighton, Proteins: Structure and Molecular Properties, W.H. Freeman & Co., San Francisco, (1983)), acetylation of the N-terminal amine, and amidation of any C-terminal carboxyl group. The modified forms of the residues fall within the scope of the present invention.

Also included herein are antibodies in which the native glycosylation pattern of the polypeptide have been altered. This means deletion of one or more carbohydrate moieties and/or adding one or more glycosylation sites that are not present in the native polypeptide chains. Protein glycosylation is typically N-linked (attached to an Asp side chain) or O-linked (attached to a hydroxyamino acid, most commonly Ser or Thr; possibly 5-hydroxyPro or 5-hydroxyLys). The tripeptide Asp-Z-Ser and Asp-Z-Thr (where Z is any amino acid but Pro) are recognition sequences for enzymatic attachment of the carbohydrate moiety to the Asp side chain. The presence of either of these sequences creates a potential N-glycosylation site. O-linked glycosylation usually involves binding of N-acetylgalactosamine, galactose, or xylose. Addition of glycosylation sites to the polypeptide may be accomplished by altering the native amino acid sequence to include e one or more of the above-described tripeptide sequences (for N-linked glycosylation sites) or addition of, or substitution by, one or more Serine or Threonine (for O-linked glycosylation sites). The amino acid sequence may be altered through changes at the DNA level, e.g., by mutating the DNA encoding the Ig polypeptide chain at preselected bases to generate codons that encode the desired amino acids. See, for example U.S. Pat. No. 5,364.934.

Chemical or enzymatic coupling of glycosides to the polypeptide may also be used. Depending on the coupling mode used, the sugar(s) may be attached to (a) Arginine and His, (b) free carboxyl groups, (c) free sulfhydryl groups such as those of Cys, (d) free hydroxyl groups such as those of Serine, Thr, or hydroxyPro, (e) aromatic residues such as those of Phe, Tyr, or Trp, or (f) the amide group of Gln. These methods are described in WO87/05330 (11 Sep. 1987) and in Aplin et al., CRC Crit. Rev. Biochem., pp. 259-306 (1981).

Removal of existing carbohydrate moieties may be accomplished chemically or enzymatically or by mutational substitution of codons (as described above). Chemical deglycosylation is achieved, for example, by exposing the polypeptide to trifluoromethanesulfonic acid, or an equivalent compound cleaves most or all sugars except the linking sugar (N-acetylglucosamine or N-acetylgalactosamine), while leaving the polypeptide intact. See: Hakimuddin et al., Arch. Biochem. Biophys., 259:52 (1987); Edge et al., Anal. Biochem. 118:131 (1981). Any of a number of endo- and exo-glycosidases are used for enzymatic cleavage of carbohydrate moieties from polypeptides (Thotakura et al., Meth. Enzymol. 138:350 (1987)).

Glycosylation at potential glycosylation sites may be prevented by the use of the tunicamycin (Duskin et al., J Biol Chem, 257:3105 (1982) which blocks formation of N-glycosidic linkages.

Another type of chemical modification of the present antibodies comprises bonding to any one of a number of different nonproteinaceous polymers, such as polyethylene glycol (PEG), polypropylene glycol, or polyoxyalkylenes, in the manner described in U.S. Pat. Nos. 4,640,835; 4,496,689; 4,301,144; 4,670,417; 4,791,192 and 4,179,337 and WO93/00109.

In addition to in vivo diagnostic and therapeutic uses, the antibodies or fragments of the present invention may be used to quantitatively or qualitatively detect the presence of Met in a cellular or other biological sample. For example, it may be desired to monitor the level of Met in the circulation or in the tissues of a subject receiving a therapeutic dose or form of the mAb. Thus, the antibodies (or fragments thereof) useful in the present invention may be employed histologically to detect the presence of Met-bearing tumor cells.

The present invention is directed in particular to a number of useful mAbs reactive against various epitopes of the Met, of HGF or the Met-HGF complex. Most preferred are mAbs specific for Met, particularly those specific for an epitope on the Met ECD.

The mAbs and combinations of the present invention, along with various names used for each mAb (some being abbreviations of longer designations) are shown in Table 1, below. The hybridomas producing these mAbs have been deposited in the American Type Culture Collection (ATCC) prior to the filing of the present application. Their ATCC Patent Deposit Designations (or accession numbers), are provided in Table 1.

TABLE 1

| | | | |
|---|---|---|---|
| mAb | | | |
| name | Hybridoma | ATCC# | Refs/Comments |

Anti-Met mAbs specific for ECD epitopes

| | | | |
|---|---|---|---|
| Met3 | 2F6-B7-A11 | PTA-4349 | Examples 1-3, Example 4 |
| | (also referred to as | | |
| | “2F6”) | | |
| | Isotype: IgG2b/κ | | |
| Met5 | 3A11-A8 | PTA-4477 | Example 5 |
| | (also referred tro as | | |
| | “3A11”) | | |
| | IgG; isotype _/_{—} | | |

Anti HGF mAbs*

| | | | |
|---|---|---|---|
| A.1 | 1C10-F1-A11 | PTA-3414 | Examples 1-3, Ref 1, Ref 2 |
| A.5 | 13B1-E4-E10 | PTA-3416 | Examples 1-3, Ref 1, Ref 2 |
| A.7 | 15D7-B2 | PTA-3413 | Examples 1-3, Ref 1, Ref 2 |
| A.10 | 31D4-C9-D4 | PTA-3412 | Examples 1-3, Ref 1, Ref 2 |

(*a neutralizing mixture consisting of all four anti-HGF mAbs was reactive with the HGF-Met pair and was used in Examples 1-3.)
Ref 1: WO 01/34650A1
Ref 2: Cao et al., Proc Natl Acad Sci USA 98:7443-7448 (2001)

Initially, nuclear imaging of Met-expressing tumors was accomplished by radioiodinating a mixture of mAbs that bind to hHGF and to the ECD (the HGF-binding domain) of hMet. See Examples 1-3, below and Hay et al., Mol Imaging, 2001,1:56-62, incorporated by reference in its entirety). The ¹²⁵I-mAb mixture was injected intravenously (i.v.) into mice bearing one of several types of tumor. One class of tumors grew by autocrine stimulation of hMet by hHGF which they expressed. Other tumors grew by autocrine-paracrine stimulation of mMet by mHGF (murine Met and murine HGF).

In addition to or combination with the nuclear imaging approach exemplified herein, the present invention also includes microscopic imaging techniques combined with immunochemical and biochemical analyses to understand the molecular bases of the observed reactions, e.g., determining the relative contributions of such parameters as total cellular Met levels, surface access of Met to mAbs, the state of Met activation, and rates of receptor turnover, to the imaging characteristics of Met-expressing tumors in vivo.

Diagnostic Compositions ands Methods

Anti-hMet mAbs alone, preferably Met3 or Met5, a combination of anti-hMet mAbs, e.g., Met3+Met5, or a combination of one or more anti-hMet mAbs with anti-hHGF mAbs, offer a novel approach in the imaging by, for example, radioimmunoscintigraphy (as well as for immunotherapy and radioimmunotherapy) of neoplasms in mammals, preferably humans. Several mAbs or derivatives thereof (e.g., Bexxar®, OncoScint®, ProstaScint®, Verluma®, CEAScan®, Zevalin®) have received clinical approval for radioimmunoscintigraphy or radioimmunotherapy. All these target neoplasms based on the cells of origin of the tumor (e.g., carcinoma, sarcoma., lymphoma, etc.). In contrast, the present invention targets neoplasms based on the inappropriate expression of Met and/or hHGF, which has been correlated with poor prognosis in a wide range of human solid tumors not limited by tissue of origin. In neoplastic cells the aberrant expression of Met and HGF leads to emergence of an invasive/metastatic phenotype.

One or a combination of Anti-hMet mAbs, optionally in combination with anti-hHGF mAbs offer a novel approach to the radioimmunoscintigraphy to immunotherapy and radioimmunotherapy of neoplasms in animals and in humans.

Several mAbs or derivatives thereof that have received clinical approval for radioimmunoscintigraphy or radioimmunotherapy (e.g., Bexxar®, OncoScint®, ProstaScint®, Verluma®, CEAScan®, Zevalin®) all target neoplasms based on the tumor's cells of origin (e.g., carcinoma, sarcoma., lymphoma, etc.). In contrast, anti-hMet mAbs alone or in combination with anti-hHGF mAbs target neoplasms based on the inappropriate expression of Met and/or hHGF, which has been correlated with poor prognosis in a wide range of human solid tumors. In neoplastic cells the aberrant expression of Met and HGF leads to emergence of an invasive/metastatic phenotype. Such radiolabeled mAbs are effective at detecting Met- and/or HGF/SF-expressing tumors in humans.

The present mAbs can be detectably labeled and used, for example, to detect Met on the surface or in the interior of a cell. Such approaches are exemplified below. The fate of the mAb during and after binding can be followed in vitro or in vivo by using the appropriate method to detect the label. The labeled mAb may be utilized in vivo for diagnosis and prognosis

The term “diagnostically labeled” means that the mAb has attached to it a diagnostically detectable label. There are many different labels and methods of labeling known to those of ordinary skill in the art. Examples of the types of labels which can be used in the present invention include radioactive isotopes, paramagnetic isotopes, and compounds which can be imaged by positron emission tomography (PET). Those of ordinary skill in the art will know of other suitable labels for binding to the mAbs used in the invention, or will be able to ascertain such, by routine experimentation. A number of such classes of diagnostic labels are disclosed below. Diagnostically-labeled (e.g., radiolabeled) mAbs are effective at detecting Met- and/or HGF-expressing human tumors in animal models and are therefore expected to be similarly effective in humans bearing such tumors.

Because of the greater expression of Met on tumor cells, it is possible to distinguish the binding of these labeled mAbs to tumors vs. normal tissue background. In addition, because of the broad expression of Met across tumor classes (i.e., different organs and tissue of origin) imaging of this single surface marker will not be specific for any particular tumor type but rather can be used in general for any Met-expressing tumor. This is in contrast to the imaging agents that target tumor type-specific markers.

Suitable detectable labels for diagnosis and imaging include radioactive, fluorescent, fluorogenic, chromogenic, or other chemical labels. Useful radiolabels, which are detected simply by gamma counter, scintillation counter, PET scanning or autoradiography include ³H, ¹²⁴I, ¹²⁵I, ¹³¹I, ³⁵S and ¹⁴C. In addition, ¹³¹I is a useful therapeutic isotope (see below).

Common fluorescent labels include fluorescein, rhodamine, dansyl, phycoerythrin, phycocyanin, allophycocyanin, o-phthaldehyde and fluorescamine. The fluorophore, such as the dansyl group, must be excited by light of a particular wavelength to fluoresce. See, for example, Haugland, Handbook of Fluorescent Probes and Research Chemicals, Sixth Ed., Molecular Probes, Eugene, Oreg., 1996). Fluorescein, fluorescein derivatives and fluorescein-like molecules such as Oregon Green™ and its derivatives, Rhodamine Green™ and Rhodol Green™, are coupled to amine groups using the isothiocyanate, succinimidyl ester or dichlorotriazinyl-reactive groups. Similarly, fluorophores may also be coupled to thiols using maleimide, iodoacetamide, and aziridine-reactive groups. The long wavelength rhodamines, which are basically Rhodamine Green™ derivatives with substituents on the nitrogens, are among the most photostable fluorescent labeling reagents known. Their spectra are not affected by changes in pH between 4 and 10, an important advantage over the fluoresceins for many biological applications. This group includes the tetramethylrhodamines, X-rhodamines and Texas Red™ derivatives. Other preferred fluorophores for derivatizing the peptide according to this invention are those which are excited by ultraviolet light. Examples include cascade blue, coumarin derivatives, naphthalenes (of which dansyl chloride is a member), pyrenes and pyridyloxazole derivatives. Also included as labels are two related inorganic materials that have recently been described: semiconductor nanocrystals, comprising, for example, cadmium sulfate (Bruchez, M. et al., Science 281:2013-2016 (1998), and quantum dots, e.g., zinc-sulfide-capped cadmium selenide (Chan, W. C. W. et al., Science 281:2016-2018 (1998)).

In yet another approach, the amino groups of a anti-Met mAb are allowed to react with a reagent that yields a fluorescent product, for example, fluorescamine, dialdehydes such as o-phthaldialdehyde, naphthalene-2,3-dicarboxylate and anthracene-2,3-dicarboxylate. 7-nitrobenz-2-oxa-1,3-diazole (NBD) derivatives, both chloride and fluoride, are useful to modify amines to yield fluorescent products.

The mAbs can also be labeled for detection using fluorescence-emitting metals such as ¹⁵²Eu+, or others of the lanthanide series. These metals can be attached to the peptide using such metal chelating groups as diethylenetriaminepentaacetic acid (DTPA) or ethylenediamine-tetraacetic acid (EDTA). DTPA in anhydride form can readily modify the NH₂-containing mAbs.

For in vivo diagnosis or therapy, radionuclides may be bound to the mAb either directly or indirectly using a chelating agent such as DTPA and EDTA. Examples of such radionuclides are ⁹⁹Tc, ¹²³I, ¹²⁵I, ¹³¹I, ¹¹¹In, ⁹⁷Ru, ⁶⁷Cu, ⁶⁷Ga, ⁶⁸Ga, ⁷²As, ⁸⁹Zr, ⁹⁰Y and ²⁰¹Tl. Generally, the amount of labeled mAb needed for detectability in diagnostic use will vary depending on considerations such as age, condition, sex, and extent of disease in the patient, contraindications, if any, and other variables, and is to be adjusted by the individual physician or diagnostician. Dosage can vary from 0.01 mg/kg to 100 mg/kg.

The mAbs can also be made detectable by coupling them to a phosphorescent or a chemiluminescent compound. The presence of the chemiluminescent-tagged peptide is then determined by detecting the presence of luminescence that arises during the course of a chemical reaction. Examples of particularly useful chemiluminescers are luminol, isoluminol, theromatic acridinium ester, imidazole, acridinium salt and oxalate ester. Likewise, a bioluminescent compound may be used to label the peptides. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

In yet another embodiment, colorimetric detection is used, based on chromogenic compounds which have, or result in, chromophores with high extinction coefficients.

In situ detection of the labeled mAb may be accomplished by removing a histological specimen from a subject and examining it by microscopy under appropriate conditions to detect the label. Those of ordinary skill will readily perceive that any of a wide variety of histological methods (such as staining procedures) can be modified in order to achieve such in situ detection.

For diagnostic in vivo radioimaging, the type of detection instrument available is a major factor in selecting a radionuclide. The radionuclide chosen must have a type of decay, which is detectable by a particular instrument. In general, any conventional method for visualizing diagnostic imaging can be utilized in accordance with this invention. Another factor in selecting a radionuclide for in vivo diagnosis is that its half-life be long enough so that the label is still detectable at the time of maximum uptake by the target tissue, but short enough so that deleterious irradiation of the host is minimized. In one preferred embodiment, a radionuclide used for in vivo imaging does not emit particles, but produces a large number of photons in a 140-200 keV range, which may be readily detected by conventional gamma cameras.

A preferred diagnostic method is radioimmunoscintigraphic analysis, which is preferably performed in a manner that results in serial total body gamma camera images and allows determination of regional activity by quantitative “region-of-interest” (ROI) analysis. Examples are provided below.

According to the present invention, every solid human tumor that is biopsied or excised can be interrogated routinely by immunohistochemistry to characterize its Met-expression status. All patients with Met-positive tumors would then undergo a Met-directed nuclear imaging study to disclose residual or clinically occult lesions and assess their abundance of Met, or to document that none are evident. Any patient with residual or newly disclosed lesions could be evaluated by provocative diagnostic MRI and/or ultrasonography to determine the physiologic responsiveness of their tumors, and an appropriate therapy regimen (chemotherapy, immunotherapy, radioimmunotherapy) would then be devised. Finally, provocative functional imaging or Met-directed nuclear imaging would be used to monitor changes in Met abundance and activity in response to therapy.

As is exemplified below, tumors growing in an autocrine manner due to interaction of hHGF and hMet took up and cleared the ¹²⁵I-mAb mixture more rapidly than did tumors expressing mHGF, mMet or both. In tumors with hHGF/hMet, the ratio of mean tumor radioactivity to total body radioactivity was >0.3 one day postinjection. Thus, radioimmunodetection of tumors undergoing autocrine-like growth due to hHGF and hMet expression is achieved using a radioiodinated (¹²⁵I) mixture of mAbs that are reactive with the ligand (HGF) -receptor (Met) pair.

The present methods offer newly diagnosed cancer patients a novel sort of “metastatic risk stratification” that uses noninvasive means to assess as high or low the probability that a given tumor will subsequently invade or metastasize, without any dependence on the tumor's “tissue” of origin. Such information improves our ability to design appropriate monitoring and therapy protocols on an individual patient basis. Very large number of patients can benefit from the present invention of using anti-hMet mAbs for diagnostic imaging and for immunotherapy and/or radioimmunotherapy.

The present inventors calculated that, for example, if only half of all patients in Michigan with newly discovered solid tumors were to undergo imaging using the present methods—with either anti-hMet mAb and/or anti-hHGF mAb—as part of their staging and metastatic risk assessment, that number, >20,000 cases per year, would far exceed the actual annual incidence of any single type of cancer in Michigan, and would far exceed the combined clinical volume currently served by all other FDA-approved mAbs.

In vivo imaging may be used to detect occult metastases which are not observable by other methods. The expression of Met can be correlated with progression of diseases in cancer patients such that patients with late stage cancer have higher levels of Met expression (or HGF binding) in both their primary tumors and metastases. Met- or HGF-targeted imaging could be used to stage tumors non-invasively or to detect another disease which is associated with the presence of increased levels of Met/HGF.

The compositions of the present invention may be used in diagnostic, prognostic or research procedures in conjunction with any appropriate cell, tissue, organ or biological sample of the desired animal species. By the term “biological sample” is intended any fluid or other material derived from the body of a normal or diseased subject, such as blood, serum, plasma, lymph, urine, saliva, tears, cerebrospinal fluid, milk, amniotic fluid, bile, ascites fluid, pus and the like. Also included within the meaning of this term is a organ or tissue extract and a culture fluid in which any cells or tissue preparation from the subject has been incubated.

The diagnostically labeled mAbs of the invention may be incorporated into convenient dosage forms Preferably, for diagnosis, the labeled mAbs are administered systemically, e.g., by injection or infusion. When used, injection or infusion may be by any known route, preferably intravenous injection or infusion, subcutaneous injection, intramuscular, intracranial or intrathecal injection or infusion, or intraperitoneal administration. Injectables can be prepared in conventional forms, either as solutions or suspensions, solid forms

The present invention may be used in the diagnosis of any of a number of animal genera and species, and are equally applicable in the practice of human or veterinary medicine. Thus, the compositions can be used with domestic and commercial animals, including birds and more preferably mammals, as well as humans.

Reagent Compositions

As noted above, the antibody compositions of this invention also additional utility to the therapeutic or in vivo diagnostic uses. For instance, the antibody compositions are useful for detecting overexpression of Met in specific cells and tissues. (This can also serve as a diagnostic tool.) Various immunoassay techniques known in the art may be used, such as competitive binding assays, direct or indirect sandwich assays and immunoprecipitation assays conducted in either heterogeneous or homogeneous phases. See, for example, Zola, Monoclonal Antibodies: A Manual of Techniques, CRC Press, Inc. (1987) pp. 147-158). The antibodies used in this manner may be detectably labeled with a detectable label that produces, either directly or indirectly, a detectable signal. Convenient labels for in vitro uses include radioisotopes, for ³H, ¹⁴C, ³²P, ³⁵S, or ¹²⁵I. Fluorescent and chemiluminescent labels and systems are described above. Any known method known for conjugating or linking a detectable label to an antibody may be used, for example, those described in Hunter et al., Nature 194:495 (1962); G. S. David et al., Biochemistry 13:1014-1021 (1974); D. Pain et al., J Immunol Meth 40:219-230 (1981); and H. Nygren, J. Histochem Cytochem. 30:407 (1982).

A preferred way to label the antibody or fragment is by linking it to an enzyme and using it in an enzyme immunoassay (EIA), or enzyme-linked immunosorbent assay (ELISA). Such assays are described in greater detail in: Butler, J. E., The Behavior of Antigens and Antibodies Immobilized on a Solid Phase (Chapter 11) In: STRUCTURE OF ANTIGENS, Vol. 1 (Van Regenmortel, M., CRC Press, Boca Raton 1992, pp. 209-259; Butler, J. E., ELISA (Chapter 29), In: van Oss, C. J. et al., (eds), IMMUNOCHEMISTRY, Marcel Dekker, Inc., New York, 1994, pp. 759-803 Butler, J. E. (ed.), IMMUNOCHEMISTRY OF SOLID-PHASE IMMUNOASSAY, CRC Press, Boca Raton, 1991; Voller, A. et al., Bull. WHO 53:55-65 (1976); Voller, A. et al., J. Clin. Pathol. 31:507-520 (1978); Butler, J. E., Meth. Enzymol. 73:482-523 (1981); Maggio, E. (ed.), Enzyme Immunoassay, CRC Press, Boca Raton, 1980 Ishikawa, E. et al. (eds.) Enzyme Immunoassay, Kagaku Shoin, Tokyo, 1981.

This enzyme, in turn, when later exposed to its substrate, will react with the substrate in such a manner as to produce a chemical moiety which can be detected, for example, by spectrophotometric, fluorometric or by visual means. Enzymes which are commonly used for this purpose include horseradish peroxidase, alkaline phosphatase, glucose-6-phosphate dehydrogenase, malate dehydrogenase, staphylococcal nuclease, Δ-V-steroid isomerase, yeast alcohol dehydrogenase, α-glycerophosphate dehydrogenase, triose phosphate isomerase, asparaginase, glucose oxidase, β-galactosidase, ribonuclease, urease, catalase, glucoamylase and acetylcholinesterase.

The antibodies of the present invention are also useful as affinity ligands for binding to Met or to cells expressing Met in assays, preparative affinity chromatography and solid phase separation of molecules from a mixture that includes Met. Such antibody compositions may also be used to identify, enrich, purify or isolate cells to which the antibodies bind, using flow cytometric and/or solid phase methodologies. The mAb may be immobilized using conventional methods, e.g. binding to CNBr-activated Sepharose® or Agarose®, NHS-Agarose® or Sepharose®, epoxy-activated Sepharose® or Agarose®, EAH-Sepharose® or Agarose®, streptavidin-Sepharose® or Agarose® in conjunction with biotinylated mAb. In general the mAbs of the invention may be immobilized by any other method which is capable of immobilizing these compounds to a solid phase for the indicated purposes. See, for example Affinity Chromatography: Principles and Methods (Pharmacia LKB Biotechnology). Thus, one embodiment is a composition comprising a mAb or mixture thereof, as described herein, bound to a solid support or a resin. The compound may be bound directly or via a spacer, preferably an aliphatic chain having about 2-12 carbon atoms.

By “solid phase” or “solid support” or “carrier” is intended any support or carrier capable of binding the mAb or derivative. Well-known supports, or carriers, in addition to Sepharose® or Agarose® described above are glass, polystyrene, polypropylene, polyethylene, dextran, nylon, amylases, natural and modified celluloses such as nitrocellulose, polyacrylamides, polyvinylidene difluoride, other agaroses, and magnetite, including magnetic beads. The carrier can be totally insoluble or partially soluble. The support material may have any possible structural configuration so long as the coupled molecule is capable of binding to receptor material. Thus, the support configuration may be spherical, as in a bead, or cylindrical, as in the inside surface of a test tube or microplate well, or the external surface of a rod. Alternatively, the surface may be flat such as a sheet, test strip, bottom surface of a microplate well, etc.

Pharmaceutical and Therapeutic Compositions and their Administration

The compounds that may be employed in the pharmaceutical compositions of the invention include all of those compounds described above, as well as the pharmaceutically acceptable salts of these compounds. A composition of this invention may be active per se, or may act as a “pro-drug” that is converted in vivo to the active form.

Effective dosages and schedules for administering the present compositions antagonist may be determined empirically; making such determinations is within the skill in the art. Those skilled in the art will understand that the effective dosage of the mAb composition will vary depending on, for example, the species of subject being treated, the route of administration, the particular type of mAb preparation or construct being used and any other drugs or agents being administered to the subject mammal. Guidance in selecting appropriate doses of mAbs is found in the literature on therapeutic uses of antibodies, e.g., Handbook of Monoclonal Antibodies, S. Ferrone et al., eds., Noges Publications, Park Ridge, N.J. (1985), particularly chap. 22 and pp. 303-357; Smith et al., Antibodies in Human Diagnosis and Therapy (Haber et al., eds.) Raven Press, New York (1977), pp. 365-389. A typical daily dosage of the therapeutic mAb compositions might range between about 1 μg and about 100 mg per kg of body weight, depending on the factors mentioned above.

In another embodiment, the present mAb composition is administered to a subject in combination with an effective amount of one or more other therapeutic agents or in conjunction with another therapeutic modality such as radiotherapy. Therapeutic agents contemplated include anticancer chemotherapeutics, immunoadjuvants and biological products such as immunostimulatory cytokines. It is believed that treatment of a subject bearing a Met-expressing tumor with an antibody composition of this invention will “sensitize” the tumor rendering it susceptible to lower doses of chemotherapeutic drugs, including levels below those that are currently considered effective by themselves (i.e., without the present antibody). Drugs intended for use in the combination therapies of the present invention include any known in the art, such as doxorubicin, 5-fluorouracil, cytosine arabinoside (Ara-C), cyclophosphamide, thiotepa, busulfan, Taxol, methotrexate, cisplatin, carbo-platin, melphalan, vinblastine, etc. The antibody composition may be administered before, after or concurrent with one or more chemo- or biotherapeutic agents. The amount of the antibody composition and the conventional drug to be used together depend, for example, on the type of drug, the nature and extent of the tumor or cancer being treated, the scheduling and the respective routes of administration. Determination of precise doses are determined empirically and based on known responses to the conventional or better-known agents. In general, the dose would generally be less than if each of the antibody composition and conventional drug were administered individually.

Following administration of the present compositions (alone or in combination), the subjects condition and the state of the tumor or cancer are be monitored in various conventional ways. For example, the tumor mass may be monitored by physical means (including palpation), by standard x-ray and other radiographic techniques, and/or by using the novel diagnostic methods and compositions described herein.

The compounds of the invention, as well as the pharmaceutically acceptable salts thereof, may be incorporated into convenient dosage forms, such as capsules, impregnated wafers, tablets or injectable preparations. Solid or liquid pharmaceutically acceptable carriers may be employed. Injectables can be prepared in conventional forms, either as solutions or suspensions, solid forms suitable for solution or suspension in liquid prior to injection, or as emulsions. Solid carriers include starch, lactose, calcium sulfate dihydrate, terra alba, sucrose, talc, gelatin, agar, pectin, acacia, magnesium stearate and stearic acid. Liquid carriers include syrup, peanut oil, olive oil, saline, water, dextrose, glycerol and the like. Similarly, the carrier or diluent may include any prolonged release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., a solution), such as an ampoule, or an aqueous or nonaqueous liquid suspension. A summary of such pharmaceutical compositions may be found, for example, in Remington's Pharmaceutical Sciences, Mack Publishing Company, Easton Pa. (Gennaro 18th ed. 1990).

Solid carriers include starch, lactose, calcium sulfate dihydrate, terra alba, sucrose, talc, gelatin, agar, pectin, acacia, magnesium stearate and stearic acid. Liquid carriers include syrup, peanut oil, olive oil, saline, water, dextrose, glycerol and the like. Similarly, the carrier or diluent may include any prolonged release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., a solution), such as an ampoule, or an aqueous or nonaqueous liquid suspension. A summary of such pharmaceutical compositions may be found, for example, in Remington's Pharmaceutical Sciences, Mack Publishing Company, Easton Pa. (Gennaro 18th ed. 1990).

The pharmaceutical preparations are made following conventional techniques of pharmaceutical chemistry involving such steps as mixing, granulating and compressing, when necessary for tablet forms, or mixing, filling and dissolving the ingredients, as appropriate, to give the desired products for oral, parenteral, topical, transdermal, intravaginal, intrapenile, intranasal, intrabronchial, intracranial, intraocular, intraaural and rectal administration. The pharmaceutical compositions may also contain minor amounts of nontoxic auxiliary substances such as wetting or emulsifying agents, pH buffering agents and so forth.

Though the preferred routes of administration are systemic the pharmaceutical composition may be administered topically or transdermally, e.g., as an ointment, cream or gel; orally; rectally; e.g., as a suppository, parenterally, by injection or continuously by infusion; intravaginally; intrapenilely; intranasally; intrabronchially; intracranially, intraaurally; or intraocularly.

Also suitable for topic application are sprayable aerosol preparations wherein the composition, preferably in combination with a solid or liquid inert carrier material, is packaged in a squeeze bottle or in admixture with a pressurized volatile, normally gaseous propellant. The aerosol preparations can contain solvents, buffers, surfactants, perfumes, and/or antioxidants in addition to the compounds of the invention.

For the preferred topical applications, especially for humans, it is preferred to administer an effective amount of the compound to an affected area, e.g., skin surface, mucous membrane, eyes, etc. This amount will generally range from about 0.001 mg to about 1 g of a given antibody per application, depending upon the area to be treated, the severity of the symptoms, and the nature of the topical vehicle employed.

Therapeutic compositions of the invention may comprise, in addition to the labeled antibodies, one or more additional anti-tumor agents, such as mitotic inhibitors, e.g. vinblastine; alkylating agents, e.g., cyclophosphamide; folate inhibitors, e.g., methotrexate, piritrexim or trimetrexate; antimetabolites, e.g., 5-fluorouracil and cytosine arabinoside; intercalating antibiotics, e.g., adriamycin and bleomycin; enzymes or enzyme inhibitors, e.g., asparaginase, topoisomerase inhibitors such as etoposide; or biological response modifiers, e.g., interferons or interleukins. In fact, pharmaceutical compositions comprising any known cancer therapeutic in combination with the labeled antibodies disclosed herein are within the scope of this invention. The pharmaceutical composition may also comprise one or more other medicaments to treat additional symptoms for which the target patients are at risk, for example, anti-infectives including antibacterial, anti-fungal, anti-parasitic, anti-viral, and anti-coccidial agents.

Therapeutic Compositions

In a preferred embodiment, the antibodies described herein are “therapeutically conjugated” or “therapeutically labeled” (terms which are intended to be interchangeable) and used to deliver a therapeutic agent to the site to which the antibodies home and bind, such as sites of primary tumor or tumor metastasis. The term “therapeutically conjugated” means that the protein is conjugated to another therapeutic agent that is physically directed to a “component” of tumor growth or invasion.

Examples of useful therapeutic radioisotopes (ordered by atomic number) include ⁴⁷Sc, ⁶⁷Cu, ⁹⁰Y, ¹⁰⁹Pd, ¹²⁵I, ¹³¹I, ¹⁸⁶Re, ¹⁸⁸Re, ¹⁹⁹Au, ²¹²At, ²¹²Pb and ²¹⁷Bi. These atoms ban be conjugated to the polypeptide directly, indirectly as part of a chelate, or, in the case of iodine, indirectly as part of an iodinated Bolton-Hunter group.

Preferred doses of the radionuclide conjugates are a function of the specific radioactivity to be delivered to the target site which varies with tumor type, tumor location and vascularization, kinetics and biodistribution of the polypeptide carrier, energy of radioactive emission by the nuclide, etc. Those skilled in the art of radiotherapy can readily adjust the dose of the labeled protein in conjunction with the dose of the particular nuclide to effect the desired therapeutic benefit without undue experimentation.

Another therapeutic approach included here is the use of boron neutron capture therapy (NCT), where a boronated antibody is delivered to a desired target site, such as a tumor, most preferably an intracranial tumor (Barth, R. F., Cancer Invest. 14:534-550 (1996); Mishima, Y. (ed.), Cancer Neutron Capture Therapy, New York: Plenum Publishing Corp., 1996; Soloway, A. H., et al., (eds), J. Neuro-Oncol. 33:1-188 (1997). The stable isotope ¹⁰B is irradiated with low energy (<0.025 eV) thermal neutrons, and the resulting nuclear capture yields α particles and ⁷Li nuclei which have high linear energy transfer and respective path lengths of about 9 and 5 μm. This method is predicated on ¹⁰B accumulation in the tumor with lower levels in blood, endothelial cells and normal tissue (e.g., brain). Such delivery has been accomplished using epidermal growth factor (Yang. W. et al., Cancer Res 57:4333-4339 (1997).

In addition to boron NCT, gadolinium, specifically ¹⁵⁷Gd appears to be particularly advantageous for use in NCT with the present antibodies. It has recently been reported (Tokumitsu, H. et al., Chem Pharm Bull 47:838-842 (1999), incorporated by reference in its entirety) that ¹⁵⁷Gd has the highest thermal neutron capture cross section (255,000 barns) among naturally occurring isotopes, 66 times larger than that of ¹⁰B; Gd neutron capture reaction releases the long range (>100 μm) prompt γ-rays, internal conversion electrons, X-rays and Auger electrons. Thus, Gd-NCT may increase the chance for photons to hit tumor cells and for electrons to damage these cell locally and intensively. Another advantage is that Gd has long been used as a MRI imaging diagnostic agent. It will be possible to integrate Gd-NCT with MRI diagnosis by using the Gd-loaded dosage forms of the present antibodies. A preferred form of Gd for labeling the antibodies of this invention for use in Gd-NCT is gadopentetic acid (Gd-DTPA).

Other therapeutic agents which can be coupled to the antibodies according to the method of the invention are drugs, prodrugs, enzymes for activating pro-drugs, photosensitizing agents, gene therapeutics, antisense vectors, viral vectors, lectins and other toxins.

The therapeutic dosage administered is an amount that is therapeutically effective, as is known to or readily ascertainable by those skilled in the art. The dose is also dependent upon the age, health, and weight of the recipient, kind of concurrent treatment(s), if any, the frequency of treatment, and the nature of the effect desired, such as, for example, anti-inflammatory effects or anti-bacterial effect.

Lectins are proteins, commonly derived from plants, that bind to carbohydrates. Among other activities, some lectins are toxic. Some of the most cytotoxic substances known are protein toxins of bacterial and plant origin (Frankel, A. E. et al., Ann. Rev. Med. 37:125-142 (1986)). These molecules binding the cell surface and inhibit cellular protein synthesis. The most commonly used plant toxins are ricin and abrin; the most commonly used bacterial toxins are diphtheria toxin and Pseudomonas exotoxin A. In ricin and abrin, the binding and toxic functions are contained in two separate protein subunits, the A and B chains. The ricin B chain binds to the cell surface carbohydrates and promotes the uptake of the A chain into the cell. Once inside the cell, the ricin A chain inhibits protein synthesis by inactivating the 60S subunit of the eukaryotic ribosome Endo, Y. et al., J. Biol. Chem. 262: 5908-5912 (1987)). Other plant derived toxins, which are single chain ribosomal inhibitory proteins, include pokeweed antiviral protein, wheat germ protein, gelonin, dianthins, momorcharins, trichosanthin, and many others (Strip, F. et al., FEBS Lett. 195:1-8 (1986)). Diphtheria toxin and Pseudomonas exotoxin A are also single chain proteins, and their binding and toxicity functions reside in separate domains of the same protein chain with full toxin activity requiring proteolytic cleavage between the two domains. Pseudomonas exotoxin A has the same catalytic activity as diphtheria toxin. Ricin has been used therapeutically by binding its toxic α-chain, to targeting molecules such as antibodies to enable site-specific delivery of the toxic effect. Bacterial toxins have also been used as anti-tumor conjugates. As intended herein, a toxic peptide chain or domain is conjugated to an antibody of this invention and delivered in a site-specific manner to a target site where the toxic activity is desired, such as a metastatic focus. Methods for chemical conjugation of toxins to antibodies or other ligands and recombinant production of toxin-containing fusion proteins are known in the art (e.g., Olsnes, S. et al., Immunol. Today 10:291-295 (1989); Vitetta, E. S. et al., Ann. Rev. Immunol. 3:197-212 (1985)).

Cytotoxic drugs that interfere with critical cellular processes including DNA, RNA, and protein synthesis, have been conjugated to antibodies and subsequently used for in vivo therapy. Such drugs, including, but not limited to, daunorubicin, doxorubicin, methotrexate, and mitomycin C are also coupled to the present antibodies and used therapeutically in this form.

In another embodiment of the invention, photosensitizers maybe coupled to the present antibodies for delivery directly to a tumor.

Therapeutic Methods

The methods of this invention may be used to inhibit tumor growth and invasion in a subject. By inhibiting the growth or invasion of a tumor, the methods are intended to inhibit tumor metastasis as well. A mammalian subject, preferably a human, is administered an amount of a therapeutic antibody composition of this invention in an amount effective to inhibit tumor growth, invasion or metastasis. The compound or pharmaceutically acceptable salt thereof is preferably administered in the form of a pharmaceutical composition as described above.

Doses of the compounds preferably include pharmaceutical dosage units comprising an effective amount of the antibody or combination of antibodies. By an effective amount is meant an amount sufficient to achieve a steady state concentration in vivo which results in a measurable reduction in any relevant parameter of disease and may include growth of primary or metastatic tumor, or a measurable prolongation of disease-free interval or of survival. For example, a reduction in tumor growth in 20% of patients is considered efficacious (Frei III, E., The Cancer Journal 3:127-136 (1997)). However, an effect of this magnitude is not considered to be a minimal requirement for the dose to be effective in accordance with this invention.

In one embodiment, an effective dose is preferably 10-fold and more preferably 100-fold higher than the 50% effective dose (ED₅₀) of the composition in an in vivo assay as described herein.

The amount of active compound to be administered depends on the precise antibody or combination selected, the disease or condition, the route of administration, the health and weight of the recipient, the existence of other concurrent treatment, if any, the frequency of treatment, the nature of the effect desired, for example, inhibition of tumor metastasis, and the judgment of the skilled practitioner.

A preferred dose for treating a subject, preferably mammalian, more preferably human, with a tumor is an amount up to about 100 milligrams of total antibody protein per kilogram of body weight. A typical single dosage is between about 1 ng and about 100 mg/kg body weight. For topical administration, dosages in the range of about 0.01-20% concentration (by weight) of the compound, preferably 1-5%, are suggested. A total daily dosage in the range of about 0.1 milligrams to about 7 grams is preferred for intravenous administration. The foregoing ranges are, however, suggestive, as the number of variables in an individual treatment regime is large, and considerable excursions from these preferred values are expected. Effective doses and optimal dose ranges may be determined in vitro or in murine models using the methods described herein.

Anti-Met mAb Characterization

Scatter Assay

Recloned hybridomas were cultured in serum-free medium. Anti-hMet mAbs in culture supernatant fractions were purified individually on a protein G affinity column, and the IgG concentration was adjusted to 2 mg/ml. Individual anti-hMet mAbs were screened for neutralizing or activating activity toward Met using the MDCK cell scatter assay. Briefly, MDCK cells were plated at 7.5×10⁴ cells/100 μl/well with or without HGF (5 ng/well) in DMEM with 5% FBS. Each anti-hMet mAb was serially diluted twofold with culture medium, and 150 μl of each successive dilution was added to the cells in 96-well plates. A rabbit polyclonal antiserum with neutralizing activity against HGF/SF (1 μl/well) was included as a Met-neutralizing control. Following overnight incubation at 37° C., cells were then stained with 0.5% crystal violet in 50% ethanol v/v for 10 minutes at room temperature, and scattering was viewed using a light microscope.

Urokinase Plasminogen Activator-Plasmin Proteolytic Assay

HGF stimulation of cells expressing Met induces expression of the serine protease urokinase (uPA) and its receptor (uPAR). uPA then cleaves plasminogen to the broader specificity protease plasmin. In this assay, we supply extra plasminogen to amplify the production of plasmin, and we also supply Chromozyme PL as a colorimetric substrate for plasmin. The process results in a colored cleavage product of Chromozyme PL, which can be quantified spectrophotometrically at 405 nm.

To perform the assay, 1500 cells/well (e.g., MDCK-II cells) are seeded in a 96-well microplate in DMEM-10% FBS. On the following day, mAbs are added at various concentrations alone or in the presence of 10 units HGF; control wells include no-HGF/no-mAb, HGF without antibody, and HGF in the presence of neutralizing anti-HGF antibodies. On the third day the cells are washed twice with DMEM lacking phenol red and are incubated for four hours with 200 μl reaction buffer (50% v/v 0.05 U/ml plasminogen in DMEM without phenol red; 40% v/v 50 mM Tris-HCl, pH 8.2; and 10% v/v 3 mM Chromozyme PL in 100 mM glycine). The supernatant fractions are then analyzed spectrophotometrically for the cleavage product at 405 nm. Of 10 anti-hMet mAbs tested so far, one completely inhibits the induction of uPA by HGF (antagonist); two induce uPA to levels comparable to those seen with HGF itself (agonists); and the others form a spectrum between these extremes.

Immunofluorescence Assay

See Examples

Nuclear Imaging Experiments

The use of anti-hMet mAb Met3 in combination with a neutralizing mixture of anti-HGF mAbs for imaging Met- and HGF-expressing tumors in vivo is described in detail in the Examples.

Articles of Manufacture and Kits

The invention also provides articles of manufacture and kits containing compositions useful for diagnosing or imaging Met-positive tumors, for treating such tumors, and for detecting, quantitating or purifying Met. The article of manufacture comprises a container with a label. Suitable containers include, for example, bottles, vials, and test tubes. The containers may be formed from a variety of materials such as glass or plastic. The container holds an active agent(s) which is a composition comprising one or more mAbs according to the invention, either anti-Met antibodies, or a combination of anti-Met and anti-HGF antibodies. The label on the container indicates that the composition is used for diagnosing, monitoring or for treating cancer, as the case may be, or preferably for diagnosing, monitoring or treating particular types of cancer or tumors that express Met or for which Met levels or turnover is diagnostic or prognostic or an effective target for therapy. In another embodiment, the label indicates that the composition is useful for detecting, quantifying or purifying Met, and may also indicate directions for either in vivo or in vitro use, such as those described above.

The kit of the invention comprises the container described above and a second container comprising a buffer or other reagent(s). The kit may further include other materials desirable from a commercial and user standpoint, including other buffers, diluents, filters, needles, syringes, and package inserts with instructions for use. The kit may also contain another anticancer therapeutic agent, such as a chemotherapeutic drug or drugs.

Having now generally described the invention, the same will be more readily understood through reference to the following examples which are provided by way of illustration, and are not intended to be limiting of the present invention, unless specified.

### EXAMPLE 1

### Materials and Methods

Reagents

¹²⁵I was purchased as NaI (480-630 mBq (13-17 mCi) per μg iodine) from Amersham Corp. (Arlington Heights, Ill.). C-28 rabbit polyclonal antibody reactive with the C-terminal portion of human Met was purchased from Santa Cruz Biotechnology, Inc.

Cell Lines and Tumors

Imaging studies were initiated with a constituted mixture of S-114 cells (NIH 3T3 cells transformed with hHGF and hMet (Rong S et al., Cell Growth Differ. 1993;4:563-569) and M-114 cells (NIH 3T3 cells transformed with mHGF and mMet). Cells were grown in DMEM containing 8% calf serum. SK-LMS-1, a human leiomyosarcoma cell line autocrine for hMet and hHGF (Jeffers M et al., Mol Cell Biol. 1996;16:1115-1125), was maintained in Dulbecco's modified Eagle's medium (DMEM) supplemented with 10% FBS. DA3, a mouse mammary carcinoma cell line expressing mMet (Firon M et al., Oncogene 2000;19:2386-2397), was grown in DMEM supplemented with 10% FBS and antibiotics.

Production and Characterization of mAbs

Anti-HGF mAbs

Production and screening of anti-HGF mAbs was described in detail in WO 01/34650A1 and Cao et al., 2001, supra (both of which are incorporated by reference). Briefly, HGF was prepared from S114 cells and mouse mAbs against this protein were produced by injecting Balb/C mice intraperitoneally (i.p.) with purified native HGF protein in complete Freund's adjuvant, followed by four additional injections of the purified protein in incomplete Freund's adjuvant. After one month, a final HGF injection was given i.p. and i.v. without adjuvant. To select animals as sources of B cells/plasma cells for hybridoma production, sera of immunized mice were tested for their ability to neutralize HGF/SF in the MDCK cell scatter assay, a conventional, art-recognized assay of the biologic activity of HGF/SF. Spleen cells from animals whose sera had neutralizing antibodies were harvested and fused with P3X63AF8/653 myeloma cells using standard techniques three days after the final immunizing injection.

Anti-Met mAbs

mAbs against hMet were produced by injecting BALB/c mice intraperitoneally (i.p.) with 5×10⁶ 121-1TH-14 cells (expressing hMet) in 0.5 ml phosphate buffered saline (PBS), followed by three additional injections of the same dose. After one month, 10⁷ Okajima cells in 0.5 ml PBS were injected i.p. into each mouse. Spleen cells, obtained four days after the final injection, were fused with P3X63AF8/653 myeloma cells using standard techniques.

Hybridoma cells were screened for reactivity to hMet by ELISA using 96 well microplates coated with 0.5 μg/ml c-Met/Fc chimeric protein. c-Met/Fc is a fusion protein of the hMet ECD with human IgG₁ H chain (purchased from R & D Systems, catalog number: 358-MT) in coating buffer (0.2M Na₂CO₃/NaHCO₃, pH 9.6, 50 μl per well) overnight at 4° C. After blocking the wells with 200 μl of blocking buffer (PBS-1% BSA) for 1 hr at room temperature or overnight at 4° C., 50 μl of hybridoma supernatant were added to wells for 1.5 hr at room temperature. Plates were washed twice in washing buffer (PBS-0.05% Tween 20). Alkaline phosphatase-coupled goat anti-mouse IgG (Sigma) was added (50 μl/well) at 1:2000 dilution and allowed to incubate for 1.5 hr at room temperature. After plates were washed four times with washing buffer, the phosphatase substrate CP-nitrophenyl phosphate (Kirkegaard & Perry Laboratories, Rockville, Md.) was added for 30 min and absorbance was measured at 405 nm. Hybridomas with strong reactivity with the c-Met/Fc protein (OD value >0.5, while negative controls <0.02) were recloned, and reactivity was confirmed by ELISA.

To characterize the mAbs by IF, S-114 cells and control parental NIH-3T3 cells in 8 well strips were fixed in either formaldehyde or acetone/methanol (1:1, v/v) for 10 min at room temperature, air dried for 10 min, and blocked with blocking buffer (PBS-1% BSA) for 30 min at room temperature. Purified anti-Met mAbs and control normal mouse IgG were diluted to 20 μg/ml with blocking buffer and added to either S-114 or control NIH 3T3 cells at 50 μl/well. After incubation at 37° C. for one hour, strips were washed three times in washing buffer (PBS-0.5% Tween 20). Cells were incubated with FITC-conjugated goat anti-mouse Ig serum at a 1:20 dilution for one hour at 37° C., followed by three washes. Samples were observed by fluorescence microscopy, and the mAb showing strongest staining on acetone/methanol-fixed S-114 cells (designated 2F6) was chosen for nuclear imaging as it had the highest apparent affinity for hMet ECD.

IgG fractions were purified from hybridoma supernatants by protein G affinity chromatography and were adjusted to a final concentration of 2 mg/ml in 0.25 sodium phosphate buffer, pH 6.8-7.0. The purified IgG fractions were stored frozen in small aliquots (50 μg) and thawed just prior to radioiodination.

For the experiments described here, equal volumes of (a) the 2F6 anti-hMet mAb and (b) a neutralizing mixture consisting of 4 anti-HGF mAbs (designated A.1, A.5, A.7 and A.10), were combined to constitute a mixture reactive with the HGF-Met pair.

Radioiodination and Injection of mAb Mixture

The final mAb mixture was radioiodinated according to instructions of the radionuclide supplier. Briefly, to 25 μg of mAb mixture in 0.1 ml of 0.25 M sodium phosphate (pH 6.8) was added 74 MBq (2.0 mCi; 20 μl) of ¹²⁵I as sodium iodide and 20 nmol (10 μl) of chloramine-T. The reactants were mixed and agitated gently for 90 sec at room temperature. The reaction was quenched by the addition of 42 nmol (20 μl) of sodium metabisulfite. ¹²⁵I-mAb was separated from unreacted ¹²⁵I by ion exchange on a small column of Bio-Rad AG 1 X8 resin, 50-100 mesh. The recovered product was stored at 4° C. and was injected within 24 hours of labeling. Radiolabeling efficiency was determined in a Beckman Gamma 8000 counter, and the proportion of protein-bound ¹²⁵I in the final product was assessed by chromatography on ITLC-SG strips (Gelman) developed in 80% aqueous methanol. Assuming complete recovery of rnAb from the labeling mixture, radiolabeling efficiency was >60%, and protein-bound radioactivity accounted for ≧85% of total activity in the final product.

Imaging Procedures and Analysis

Animals were imaged and scintigrams were analyzed by methods described by the present inventors and their colleagues (Gross M D et al. (1984) Invest Radiol 19:530-534; Hay RV et al. (1997) Nucl Med Commun 18:367-378). In brief, each mouse received the ¹²⁵I-mAb mixture, 50-100 μCi (1.8-3.7 MBq) in ≦0.2 ml intravenously (i.v.) into the lateral by tail vein under light inhalation anesthesia.

Just prior to each imaging session each mouse was given up to 13 mg/kg xylazine and 87 mg/kg ketamine s.c. in the interscapular region. Anterior (for DA3 tumor-bearing mice) or posterior (for all other mice) whole-body gamma camera images of each mouse were acquired at one hour following ¹²⁵I-mAb mixture injection and again at one day, three days, and five days postinjection. Sedated mice were placed singly or in pairs on top of an inverted camera head with a protective layer over the collimator, and taped to the layer to maintain optimum limb extension. Images of ¹²⁵I activity were acquired on a Siemens LEM Plus mobile camera with a low-energy, high-sensitivity collimator. Images were acquired over a period of 15 minutes, during which between 2×10⁵ and 3×10⁶ counts were acquired per total body image.

Relative activity was determined by computer-assisted region-of-interest (ROI) analysis for each tumor, for total body, and for appropriate background regions at each imaging time point. These data are expressed below as background- and decay-corrected activity ratios. Graphical and statistical analysis of the converted data was performed with Microsoft Excel.

### EXAMPLE 2

### Characterization of Anti-Met mAb by Immunofluorescence

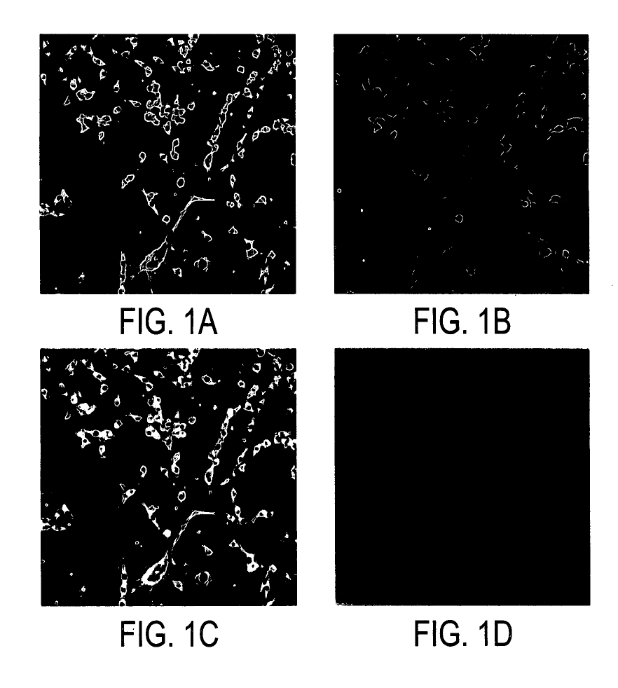

The mAb specific for the anti-hMet ECD (2F6) was characterized for IF with S-114 cells expressing hMet. Results are shown in FIG. 1. S-114 cells fixed in acetone/methanol were stained with both mAb 2F6 (=Met3) (in green, panel A) and the rabbit polyclonal antibody against the Met C-terminal peptide antibody C-28 (in red, panel B). Colocalization of staining (yellow) is evident in panel C. A Nomarski image is provided (panel D) to show the unstained location and characteristics of the cells in culture.

### EXAMPLE 3

### Image Analysis and Quantitation

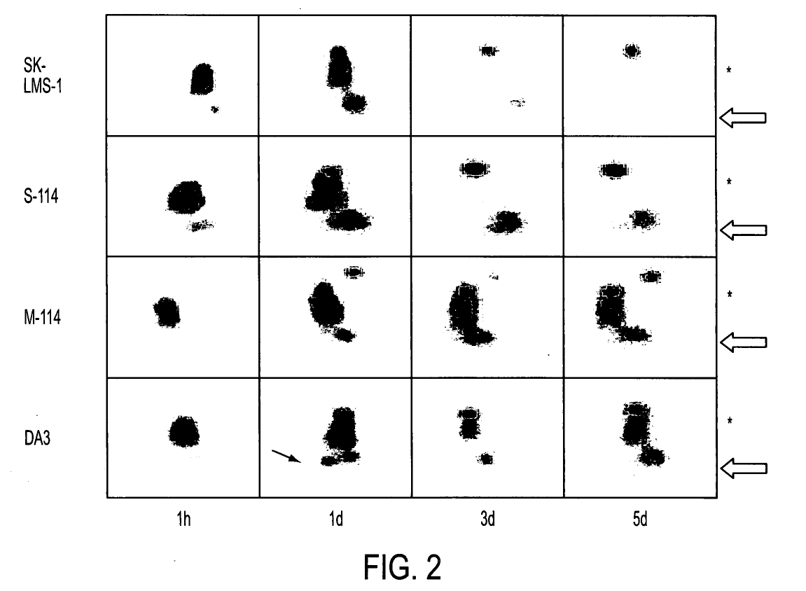

Serial total body gamma camera images of individual tumor-bearing mice were obtained between one hour and five days following i.v. injection of the ¹²⁵I-mAb mixture reactive with hHGF and hMet. See FIG. 2. Activity was evident in the human tumors (SK-LMS-1 and S-114, both of which express hHGF and hMet) as early as one hour postinjection and prominently thereafter.

Activity was also clearly seen as early as one day postinjection in murine tumors (M-114, which expresses mHGF and mMet, and DA3, expressing mMet alone). Nevertheless, mice bearing human tumors cleared radioactivity more rapidly from the circulation than mice bearing murine tumors, as evidenced by their much lower levels of visceral radioactivity at three and five days postinjection and more conspicuous thyroid activity (reflecting uptake of free radioiodine released from labeled mAbs). Even though the absolute radioactivity levels in human and murine tumors generally appeared to be comparable over time, the proportion of nonthyroidal total body radioactivity associated with human tumors—i.e., the tumor imaging contrast—appears to be greater than that associated with murine tumors at all imaging time points.

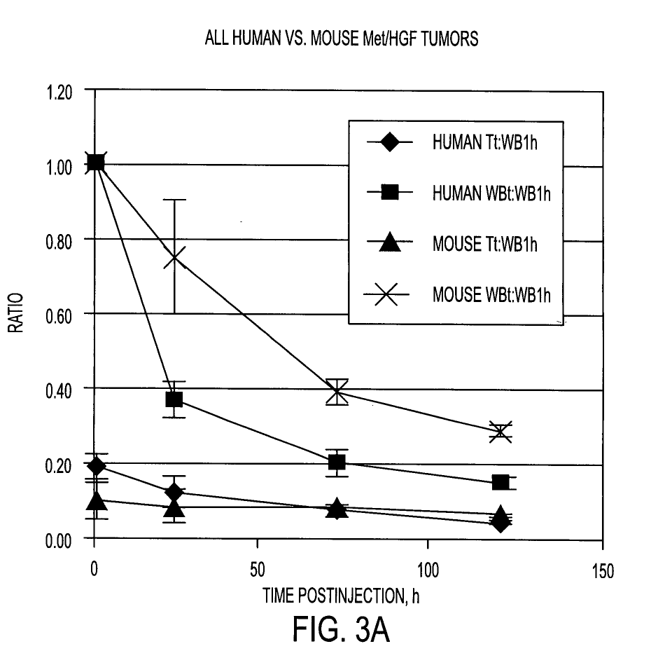

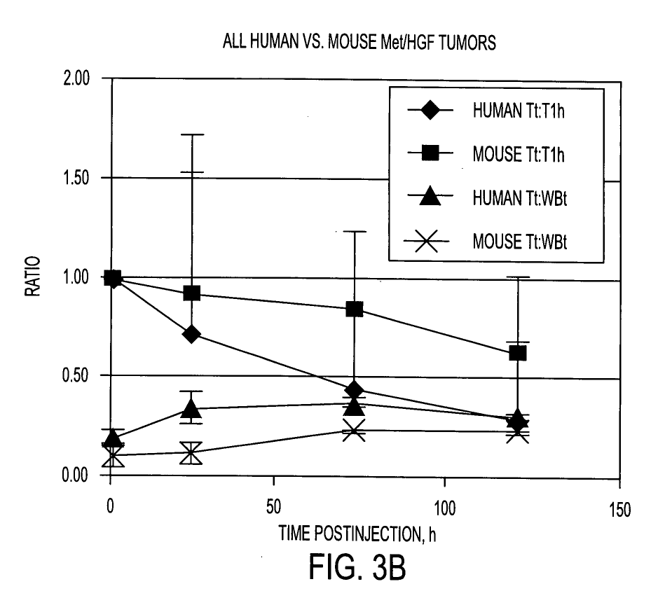

Images from four mice bearing human tumors and from three mice bearing murine tumors were assessed by ROI analysis to quantify these apparent differences and to determine whether they might be statistically significant. The results are summarized in FIGS. 3A and 3B. Indeed, t-test comparison of the mean ratio of tumor activity to total body activity (including thyroid), designated Tt:WBt, was significantly higher for human than for murine tumors at all imaging time points (p<0.02 at one hour; p≦0.001 after one hour), reaching mean values for these small groups of animals of 0.34 vs. 0.11 at one day and of 0.37 vs. 0.23 at three days postinjection. Mean retention of total body radioactivity, expressed as WBt:WB1h, was also significantly lower after one hour for human tumors (p≦0.001). Finally, although the mean retention of tumor-associated activity (Tt:T1h) was lower in human than in murine tumors after one hour postinjection, this difference was not statistically significant given the small number of animals studied (p=0.3 at one day; p<0.08 at three and five days).

ROI results were expressed as activity ratios rather than as the more traditional “percent of injected activity (% IA) (Hay et al., supra) in order to minimize the effects of variations in the efficiency of i.v. injection of radiolabeled mAb on the data. Technical factors make this variation potentially much greater in mice than in larger animals in which vascular access is easier. In this way, each animal's actual measured total body activity at the earliest imaging point serves as its own injection standard, rather than relying on a less accurate mean value for presumed injected radioactivity. Moreover, assuming that no significant radionuclide excretion occurs during the first hour postinjection, the ratio of tumor activity to total body activity at one hour (T1h:WB1h) closely approximates % IA for a tumor at one hour, and the ratio Tt:WB1h similarly approximates % IA for a tumor at time t.

Negative and positive control studies clarified the specificity of the ¹²⁵I-mAb mixture's association with murine and human tumors, and are summarized below:

1. Murine tumors (M-114 and DA3) did not show significant activity above that of blood pool by one hour or 24 hours postinjection.

2. An “aged” batch of the anti-Met and anti-HGF ¹²⁵I-mAb mixture (refrigerated for longer than one week and then repurified to remove liberated iodide) did not show significant activity above blood pool in M-114 by one hour or 24 hours postinjection. “Aged” ¹²⁵I-anti-Met mAb alone was not effective for imaging SK-LMS-1.

3. Tumor imaging experiments using freshly labeled anti-Met mAbs and anti-HGF mAbs separately indicate that both anti-Met and anti-HGF/SF contributed to the overall tumor-associated activity observed with the ¹²⁵I-mAb mixture.

Taken together, these results argue that the levels and temporal patterns of tumor-associated activity observed in this study are somehow particular to the use of freshly radioiodinated anti-Met and anti-HGF, and not to some nonspecific property of radioiodinated proteins in general.

### DISCUSSION OF EXAMPLES 1-3

The findings above demonstrate that tumors expressing hHGF and hMet (in an autocrine manner), generally a property of rapidly growing tumors, can be imaged with an ¹²⁵I-labled mixture of mAbs reactive against the HGF-Met pair. Tumors expressing mHGF and/or mMet can also be imaged with the radioiodinated mAb mixture, presumably because of epitope crossreactivity. However, in vivo metabolism of the ¹²⁵I-mAb mixture by human and murine tumors differ in their kinetics as well by other quantitative criteria. In brief, the human tumors evaluated display rapid uptake and rapid clearance of the mAb mixture from the circulation, and constitute a significantly higher proportion of total body radioactivity at times ranging from one hour to five days postinjection than do the murine tumors. Indeed, such differences would be expected between high-affinity, high-capacity tumors and those with lower affinity for binding and lower capacity for metabolizing a given radiotracer.

The imaging studies above were initiated with a “constituted” mixture of mAbs reactive with the HGF-Met pair, rather than a mAb with single epitopic specificity. This was done because of the absence of any a priori reason to select one target epitope over any other in a tumor model that expresses both a receptor (Met) and its ligand (HGF).

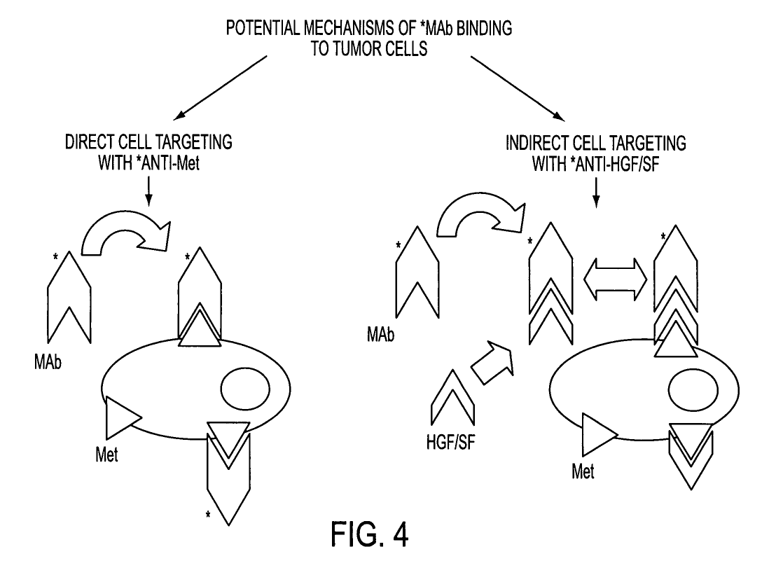

Moreover, it was already known that the various anti-HGF mAbs used in these studies bind to different epitopes. As depicted in FIG. 4 in cartoon form, radiolabeled anti-Met mAbs should bind directly to Met molecules expressed on the tumor cell surface, while anti-HGF mAbs can either bind to HGF molecules concentrated locally in the immediate vicinity of a Met-expressing cell or can form a ternary complex with HGF and Met, effectively targeting Met-expressing tumor cells indirectly, for example, by binding to Met-bound HGF.

This particular neutralizing mixture of anti-HGF mAbs may be involved in stabilizing Met so that an anti-Met mAb binds more readily or more avidly than it would otherwise. It is also possible that any one of the mAbs included in this mixture can alone be used to image these tumors.

Based on the foregoing, it is expected that newly developed radiolabeled mAbs capable of detecting Met- and/or HGF-expressing tumors in humans, will be useful as a clinical tool to obtain for a given subject, his “metastatic risk stratification” based on noninvasive assessment of the likelihood (e.g., high or low) that a given tumor will later invade and metastasize. Such information will improve our ability to design appropriate monitoring and therapy protocols on an individual patient basis.

### EXAMPLE 4

### Radioimmunoscintigraphy of hMet-Expressing Tumor Xenografts using Met3

The ability of anti-Met mAbs from a single hybridoma clone—designated Met3—were examined for their ability to image human Met-expressing tumors of four different tissue origins, and to distinguish them according to their relative abundance of Met.

¹²⁵I was purchased as NaI (480-630 MBq; 13-17 mCi per μg iodine) from Amersham Corp. (Arlington Heights, Ill.). C-28 rabbit polyclonal antibody reactive with the C-terminal portion of human Met and H-235 rabbit polyclonal antibody reactive with β-tubulin were purchased from Santa Cruz Biotechnology, Inc. The Alexa 488-conjugated anti-mouse antibody was purchased from Molecular Probes. Immunodecoration reagents were purchased from Amersham Pharmacia BioTech.

Cell Lines and Tumor Induction

S-114 cells are NIH 3T3 cells transformed with human HGF/SF and human Met (Rong et al., supra). SK-LMS-1/HGF cells are a human leiomyosarcoma cell line autocrine for human Met and human HGF/SF (Jeffers et al., supra). PC-3 cells are a human prostate carcinoma cell line. M14-Mel and SK-MEL-28 are human melanoma cell lines. All these cell lines were all maintained in DMEM supplemented with 10% FBS.

Female athymic nude (nu/nu) mice at about six weeks of age received subcutaneous injections of S-114, SK-LMS-1/HGF, or PC-3 cell suspensions in the posterior aspect of their right thighs, or of melanoma cell suspensions in the right flank adjacent to the thigh. Each mouse received between 2×10⁵ and 5×10⁵ cells. Tumors developed for 1-6 weeks before imaging, reaching ≧0.5 cm in greatest dimension by external caliper measurement. Mice were housed in small groups and given ad libitum access to mouse chow and drinking water under conditions approved by the institutional animal care committees.

Analysis of Met Expression by Cell Lines

The cultured cell lines listed above were analyzed for relative abundance of Met by immunoblotting with minor modifications of the procedures described previously (Webb, C. P. et al., 2000, Cancer Res., 60:342-349). In brief, cells were grown to near-confluency in DMEM supplemented with 10% FBS. Cell lysates were prepared, clarified, and assayed for protein concentration. Normalized aliquots of cell lysates were subjected to SDS-polyacrylamide gel electrophoresis, electrotransfer, and sequential immunodecoration with C-28 anti-Met polyclonal antibody and with anti-β-tubulin polyclonal antibody. Immune complexes were identified by enhanced chemiluminescence and visualized by exposure to X-ray film.

Preparation and Characterization of Met3

mAbs against the extracellular domain of human Met were produced and screened for reactivity as described above. Antibodies from the hybridoma clone 2F6 were identified as exhibiting the highest affinity for Met by ELISA and the highest apparent affinity for the human Met extracellular domain by IF. The antibodies from clone 2F6, used for the experiments described here, are designated Met3.

Immunohistochemical analysis of Met expression and distribution in formalin-fixed, paraffin-embedded sections of human tissues was performed as described in Knudsen et al., supra, modified as follows: Tissue sections on microscope slides were incubated with Met3 and processed with the Ventana® automated system. Slides were examined by conventional light microscopy.

Immunofluorescence analysis of Met expression in cultured cells was performed essentially as described above, incubating fixed cell monolayers with Met3 followed by FITC-conjugated anti-mouse IgG and with C-28 polyclonal antibody followed by rhodamine-conjugated anti-rabbit IgG, and visualizing staining patterns with appropriate fluorescence optics and filter sets.

Fluorescence-activated cell sorting (FACS) analysis of Met3 binding to cultured human prostate carcinoma cell lines was performed with a Becton Dickinson FACS Calibur instrument. Cultured cells were grown to near-confluency, detached and dissociated by chelation, and resuspended at about 10⁶ cells/0.1 ml in BSA-containing buffer. The cell suspensions were incubated with Met3 (10 μg/ml) for 30 minutes at 4 C, washed thrice, incubated with secondary antibody (anti-mouse Alexa green, Molecular Probes) for 15 minutes at 4 C and washed thrice before analysis.

For nuclear imaging experiments, IgG fractions were purified from 2F6 (Met3) hybridoma cell line supernatant fractions by protein G affinity chromatography and adjusted to a final concentration of 2 mg/ml in 0.25 sodium phosphate buffer, pH 6.8-7.0. The purified IgG fractions were stored frozen in small aliquots (25-50 μg) and thawed just prior to radioiodination.

Radioiodination and Injection of Met3

Met3 was radioiodinated by the procedure described above. The recovered product was stored at 4° C. until used, and injected within 24 hours of labeling. Radiolabeling efficiency was determined in a Beckman Gamma 8000 counter, and the proportion of protein-bound ¹²⁵I in the final product was assessed by chromatography on ITLC-SG strips (Gelman) developed in 80% aqueous methanol. Assuming complete recovery of mAb from the labeling mixture, radiolabeling efficiency was >60%, and protein-bound radioactivity accounted for ≧90% of total activity in the final product.

Imaging Procedures and Analysis

Animals were imaged and scintigrams were analyzed by methods described above and in Gross et al., supra; Hay et al., 1997, supra; and Hay et al., 2002, Nucl. Med. Commun. 23:367-372. In brief, each mouse received ¹²⁵I-Met3, 50-100 μCi (1.8-3.7 MBq) in ˜50 μl intravenously by tail vein injection under light inhalation anesthesia. Just prior to each imaging session each mouse was given up to 13 mg/kg xylazine and 87 mg/kg ketamine subcutaneously in the interscapular region. Posterior whole-body gamma camera images of each mouse were acquired beginning at one to two hours following ¹²⁵I-Met3 injection and again at one day, three days, and at least five or six days postinjection. Sedated mice were placed singly or in pairs on top of an inverted camera head with a protective layer over the collimator, and taped to the layer to maintain optimum limb extension. Images of ¹²⁵I activity were acquired on a Siemens LEM Plus mobile camera with a low-energy, high-sensitivity collimator. Acquisitions were obtained over a period of 15 minutes, during which we collected between 2×10⁵ and 3×10⁶ counts per total body image.

Relative activity was determined by computer-assisted region-of-interest (ROI) analysis for each tumor, for total body, and for appropriate background regions at each imaging time point. These data are expressed below as background- and decay-corrected activity ratios. Graphical and statistical analysis of the converted data utilized the program Excel (Microsoft).

### Results

Characterization of Met3

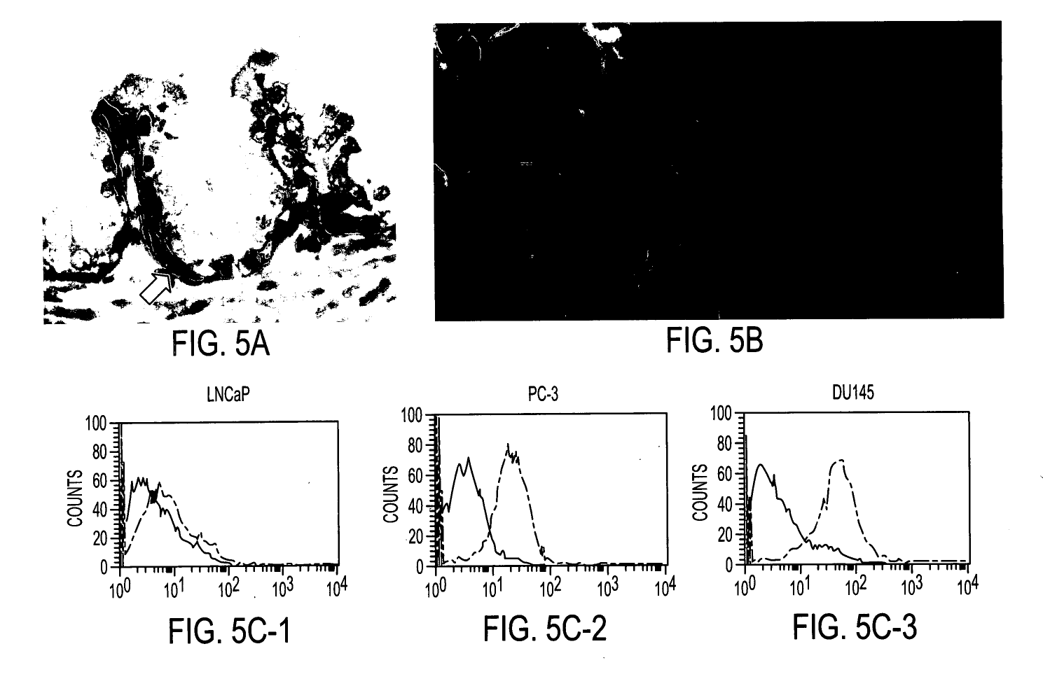

As shown herein, Met3 colocalizes with the commercially available polyclonal anti-Met antibody C-28 in cultured S-114 cells, a murine cell line transformed with human Met and human HGF/SF. FIG. 5A shows that Met3 may also be used for immunohistochemistry of human tissues, e.g., prostate tissue, in formalin-fixed, paraffin-embedded tissue sections. FIG. 5B shows that the pattern of staining for Met3 by IF analysis in primary cultures of human prostate epithelial cells replicates that observed with C-28. Moreover, Met3 binds to the surfaces of PC-3 and DU145 human prostate carcinoma cell lines, both of which express Met, but not to any significant level to the surface of LNCaP cells that express very little Met (Knudsen et al., supra). See FIG. 5C.

Analysis of Met Expression by Cell Lines

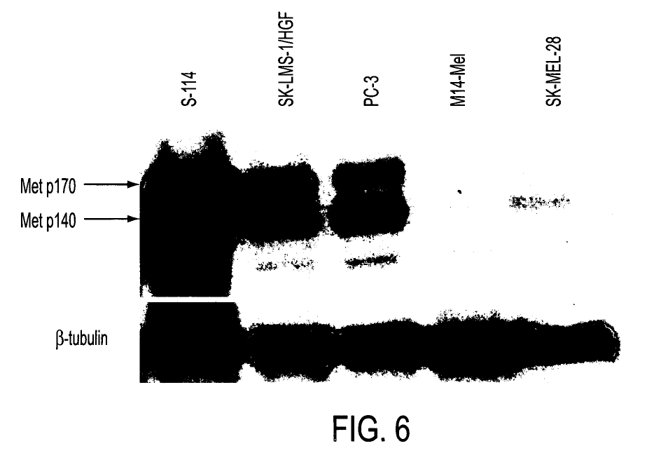

As illustrated in FIG. 6, the cell lines selected for this study vary dramatically in their relative expression of Met when cultured in the presence of serum. Cell lysates normalized to the concentration of cell protein were subjected to electrophoresis, electrotransfer, and immunodecoration with C-28 to assess the abundance of Met, and with anti-β-tubulin (as a control to verify comparable levels among the various cell lines of an irrelevant housekeeping gene product). Under these conditions, S-114 showed the highest abundance of Met, both as p170 precursor and mature p140 forms. The melanoma cell lines expressed very low levels of Met, with M14-Mel lower than SK-MEL-28. SK-LMS-1/HGF and PC-3 cells exhibited intermediate abundance of Met, with comparable levels of total Met (p170 plus p140), but with a lower ratio of p170 to p140 detected in PC-3 cells.

Image Analysis and Quantitation

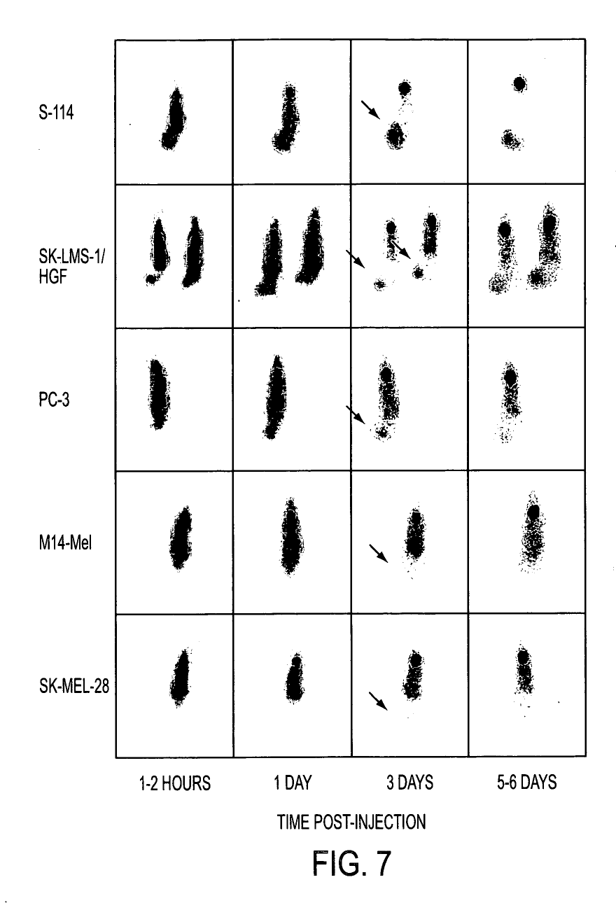

FIG. 7 shows serial total body gamma camera images of individual xenograft-bearing mice obtained between one to two hours and five to six days following i.v. injection of ¹²⁵I-Met3. A pair of simultaneously imaged host mice is depicted for SK-LMS-1/HGF xenografts. Activity is clearly visualized in the S-114 and SK-LMS-1/HGF xenografts at the earliest imaging session, with a faint asymmetry of hindlimb activity suggested initially in PC-3 xenografts. Tumor-associated radioactivity as a function of total body activity is most prominent in these three xenograft types by the third day postinjection. Neither melanoma xenograft exhibited any qualitatively appreciable uptake or retention of radioactivity during the imaging sequence.

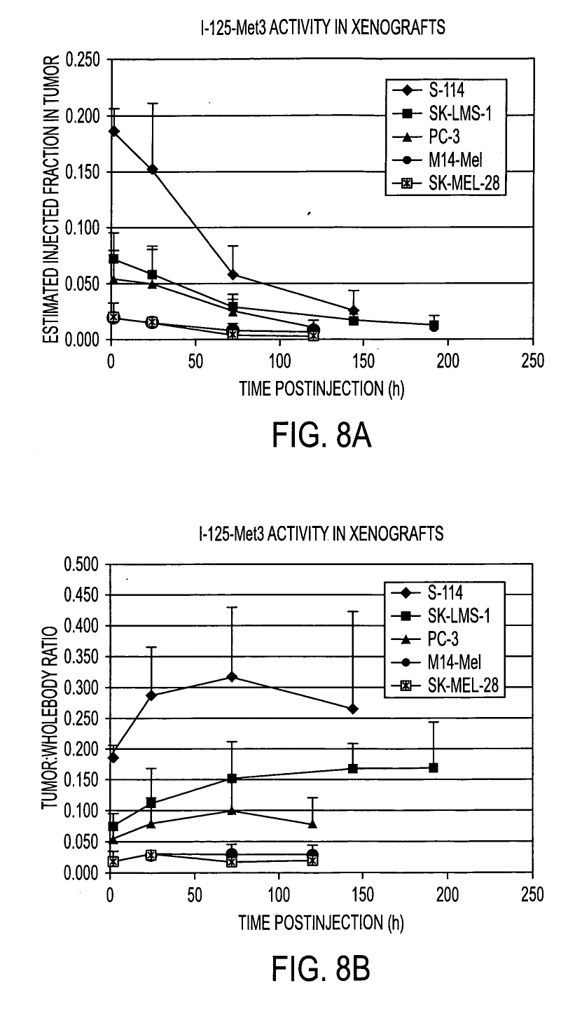

FIG. 8 shows graphical results of quantitative image ROI analysis, expressed in two forms. The upper panel displays the estimated fraction of injected activity associated with xenografts of differing tissue origin as a function of time postinjection. Each xenograft type exhibited the highest mean value for this function at the earliest imaging session, with respective maxima (±1 s.d.) of 18.6±2.1, 7.2±2.2, and 5.4±2.6% of the estimated injected activity for S-114, SK-LMS-1/HGF, and PC-3. The lower panel displays the mean ratios of tumor-to-total body activity as a function of time postinjection. For each xenograft type, the highest value for this function occurred at three days postinjection, with respective mean values (±1 s.d.) of 0.32±0.13, 0.15±0.06, and 0.10±0.04 for S-114, SK-LMS-1/HGF, and PC-3. M14-Mel or SK-MEL-28 accounted for ≦3% of injected or total body activity at any time postinjection.

### Discussion

As described in Examples 1-3, a mixture of mAbs recognizing multiple epitopes of the human Met-HGF receptor-ligand complex can be used for radioimmunoscintigraphy of autocrine tumor xenografts. These observations are extended in Example 4 which demonstrates that Met3, the product of a single hybridoma clone that recognizes a single epitope of the ECD hMet, is similarly effective for nuclear imaging. These studies further indicate that Met3 is useful for routine immunohistochemical analysis of formalin-fixed, paraffin-embedded sections of human tissue, for IF analysis of primary human cell cultures, and for FACS-based analyses of human tumor cells, in particular for the evaluation of samples of normal and malignant human prostate tissues.

The results presented here, along with additional examples, confirm that radiolabeled Met3 images Met-expressing human tumor xenografts of differing tissue origins. Moreover, the rank order of ¹²⁵I-Met3 uptake and retention levels exhibited by different types of xenografts in vivo correlates directly with the rank order of relative Met abundance as assessed biochemically in the respective parent cell lines cultured in the presence of serum. Stated another way, based on these findings, it is possible to divide, arbitrarily, tumors into categories of high, low and intermediate Met3 uptake by nuclear imaging analysis and to infer that those respective categories reflect high, low, and intermediate abundance of Met in the tumor cells.

The two tumor xenograft types that fall in the intermediate Met3 uptake category, SK-LMS-1/HGF and PC-3, show no statistically significant differences with regard to either of the ROI analysis functions exemplified here, and by immunoblotting analysis of cultured cell lysates, appear to have comparable total Met abundance (p170+p140). Nevertheless, both ROI analysis functions tended toward higher values in SK-LMS-1/HGF than in PC-3, perhaps due to the autocrine-mediated turnover of Met in the former. Thus, even minor differences in radiolabeled Met3 uptake and retention in vivo by cells with comparable total Met abundance may be attributable to differing rates of biological turnover of Met (Webb et al., supra; Jeffers, M et al., 1997, Mol. Cell. Biol. 17: 799-808).

This possibility is supported by the present inventors' recent studies comparing rates of ¹²⁵I-anti-Met mAb clearance by additional types of xenografts in vivo (see Example 55) with their responsiveness to HGF stimulation in vitro.

It is concluded that the radioiodinated anti-Met mAb designated Met3 is useful for imaging hMet-expressing xenografts of different tissue origin. According to these results, scintigraphy with radiolabeled Met3 can distinguish human tumor xenografts according to their levels of Met expression.

### EXAMPLE 5

### Radioimmunoscintigraphy of hMet-Expressing Tumor Xenografts Using a New mAb, Met3

A second anti-Met monoclonal antibody product from a single hybridoma clone, designated Met5 (see Table 1) was produced and screened essentially as described above for Met3. Immunoprecipitation and immunoblotting analysis and FACS analysis indicates that the Met5 mAb binds both canine Met and human Met. Results now shown indicate that Met5 binds to a different epitope of the ECD of Met than does the Met3 mAb.

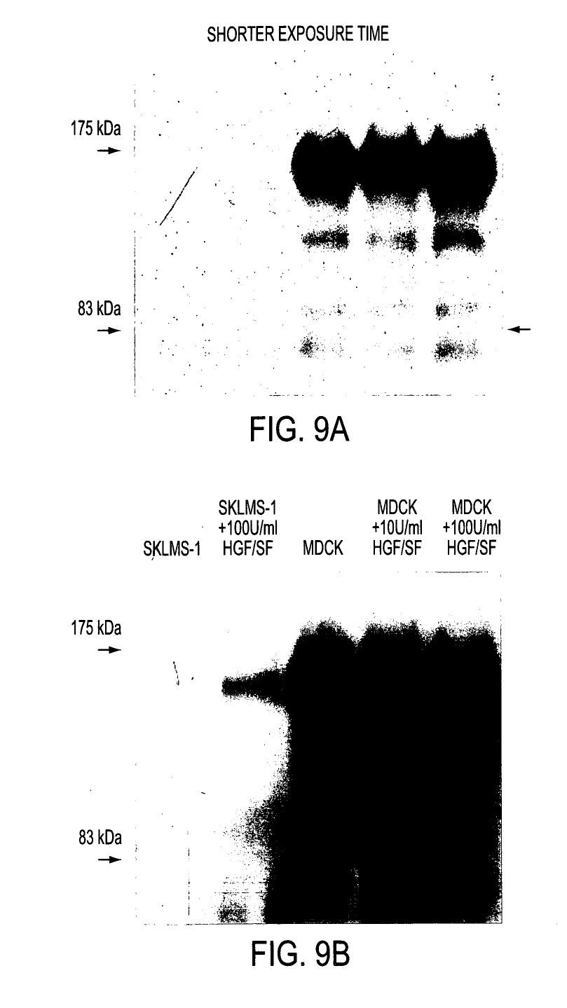

The results are shown in FIGS. 9-13.

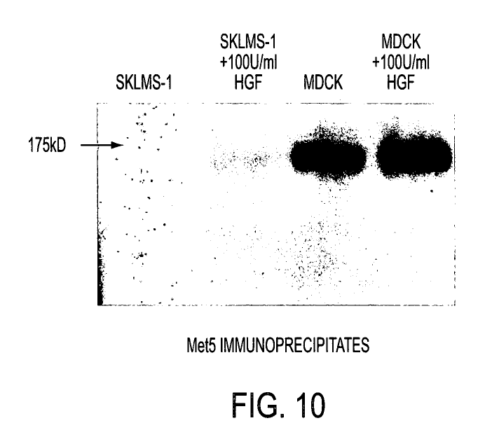

Met was found to be present on canine cells. Cells of the canine kidney cell line MDCK were cultured and exposed to HGF at the indicated concentrations. Cell lysates were prepared and immunoprecipitated with Met5 followed by electrophoresis, electrotransfer, and immunodecoration with anti-PY 4G10 (anti-phosphotyrosine antibody)to detect activated (phosphorylated) Met. SKLMS-1 cells were similarly processed as a known positive control (Met-positive, HGF-responsive). Results show the presence of a large amount of Met present in these treated cells, which increased with as stronger HGF stimulus (FIGS. 9A, 9B). This was shown in a second experiment presented in FIG. 10.

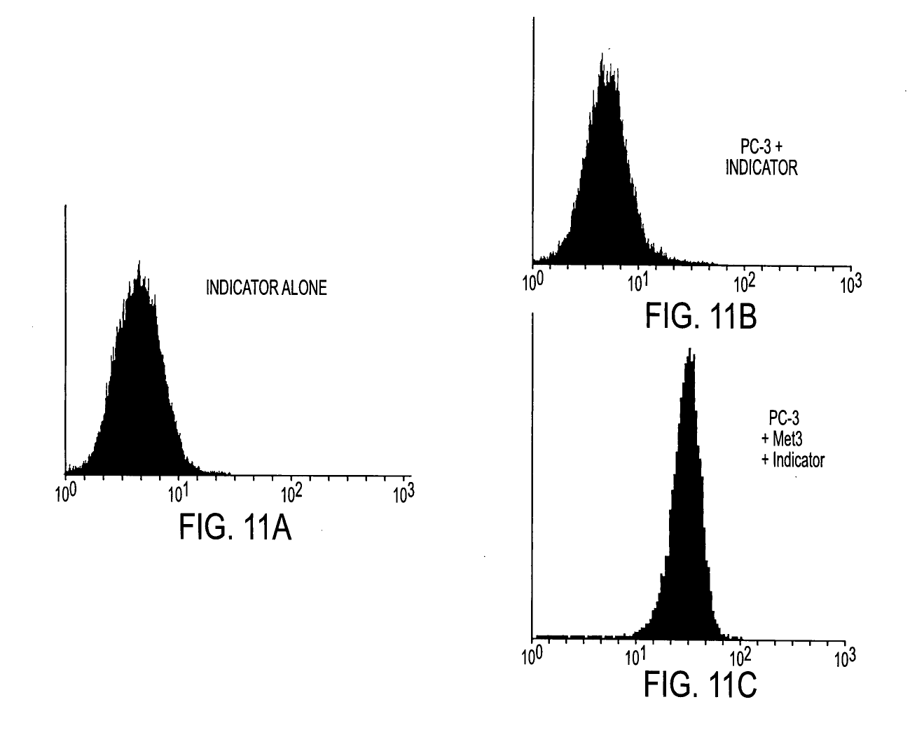

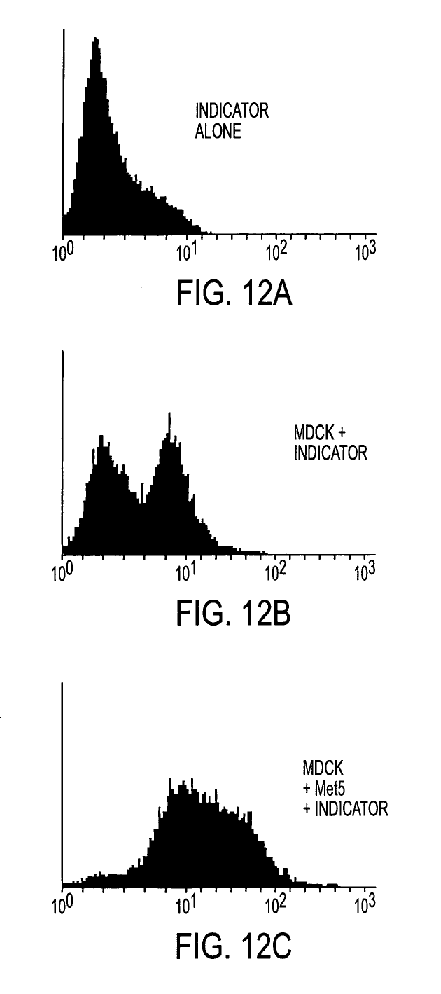

FACS analysis of Met3 binding to PC-3 human prostate carcinoma cells shows a shift of fluorescent indicator (dye-conjugated anti-mouse Ab) in the presence of Met3 to larger particle size that reflects association with cells (FIGS. 11A-11C). A similar analysis of Met5 binding (FIGS. 12A-12C) to MDCK canine kidney cells show a similar shift of the fluorescent indicator (fluorescently labeled anti-mouse antibody) to larger particle size.

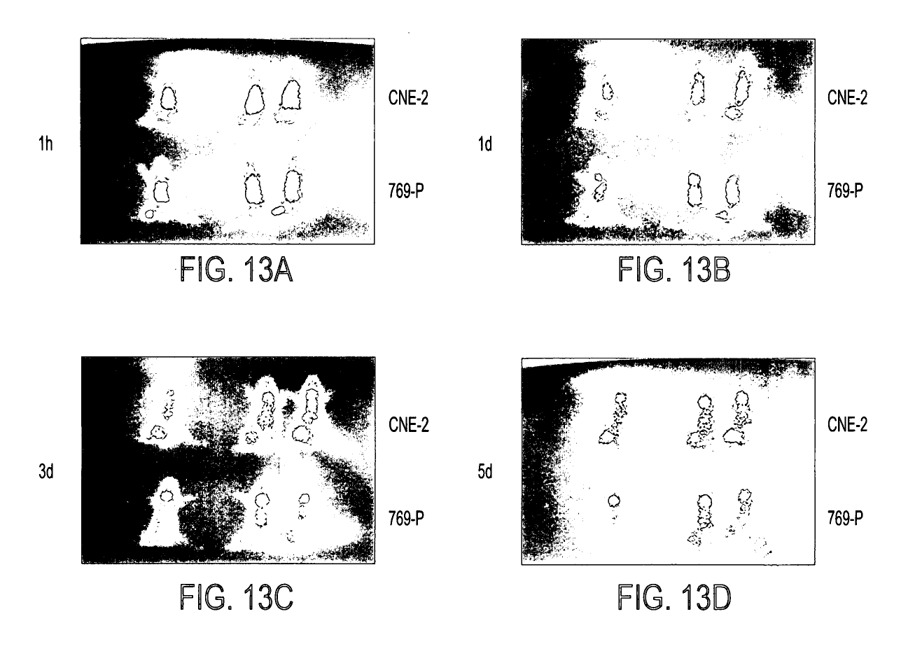

Nuclear imaging of two different types of human tumor xenografts with ¹²⁵I-Met5 is shown in FIGS. 13A-13D. Xenografts of the human nasopharyngeal carcinoma (NPC) cell line CNE-2 and the renal cell carcinoma (RCC) cell line 769-P were grown subcutaneously in the right thighs of nude mice (3 mice/group). Each mouse was injected i.v. with ¹²⁵I-Met5, and serial gamma camera images were obtained (1 hour to 5 days postinjection). Arrows appended to the image of one mouse in each group indicate the subcutaneous (thigh) tumor locations. The difference in the dynamics of antibody binding and clearance are evident. The RCC tumor cells are detected as soon as 1 hour and evidence of antibody labeling the subcutaneous tumor is gone by 3 days. In contrast, the NPC cells show labeling at day 1 and the tumors remain labeled at day 5. This may reflect turnover or internalization of the cell surface Met molecules, either inherently or in response to binding by this divalent antibody.

Thus, radioiodinated Met5, like Met3, is effective for imaging human tumor xenografts in nude mice. This reagent will permit Met-directed imaging and development of diagnostic and therapeutic agents for both humans as well as in pet dogs in which spontaneously occurring cancers of the prostate and bone are relatively common.

The references cited above are all incorporated by reference herein in their entirety, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

1. A monoclonal antibody selected from the group consisting of:
(a) a monoclonal antibody Met3 produced by the hybridoma cell line deposited in the American Type Culture Collection under Accession Number PTA-4349; and
(b) a monoclonal antibody Met5 produced by the hybridoma cell line deposited in the American Type Culture Collection under Accession Number PTA-4477,
or an antigen binding fragment or derivative of said antibody.

2-3. (canceled)

4. A monoclonal antibody, or antigen-binding fragment or derivative thereof, that has all the identifying biological characteristics of the monoclonal antibody, fragment or derivative of claim 1.

5. (canceled)

6. A humanized monoclonal antibody specific for Met, wherein
(a) the heavy chain and/or light chain variable region of said antibody, or an antigen binding site of said variable regions, has all the identifying biological or structural characteristics of the corresponding regions or sites of the monoclonal antibody of claim 1; and
(b) substantially all the remainder of the humanized monoclonal antibody is of human origin,
or an antigen binding fragment or derivative of said humanized monoclonal antibody.

7. A human monoclonal antibody specific for Met that binds to the same epitope as the epitope to which the monoclonal antibody of claim 1 binds, or an antigen binding fragment or derivative of said human antibody.

8. (canceled)

9. A composition comprising the monoclonal antibody, fragment or derivative of claim 1.

10-11. (canceled)

12. The composition of a claim 9, further comprising one or more additional antibodies specific for a Met epitope, or comprising an antigen-binding fragment or derivative of said additional one or more antibodies.

13. The composition of claim 9 further comprising one or more antibodies specific for hepatocyte growth factor (HGF), or comprising an antigen-binding fragment or derivative of said one or more HGF-specific antibodies.

14. The composition of claim 13 wherein the one or more antibodies specific for HGF is selected from the group consisting of:
(a) a monoclonal antibody produced by the hybridoma cell line deposited in the American Type Culture Collection under Accession Number PTA-3414;
(b) a monoclonal antibody produced by the hybridoma cell line deposited in the American Type Culture Collection under Accession Number PTA-3416;
(c) a monoclonal antibody produced by the hybridoma cell line deposited in the American Type Culture Collection under Accession Number PTA-3413; and
(d) a monoclonal antibody produced by the hybridoma cell line deposited in the American Type Culture Collection under Accession Number PTA-3412.

15. A diagnostically useful composition comprising
(a) the monoclonal antibody, fragment or derivative of claim 1 which is diagnostically or detectably labeled;
(b) a diagnostically acceptable carrier or excipient.

16. A diagnostically useful composition comprising
(a) the composition of claim 9 which is diagnostically or detectably labeled; and
(b) a diagnostically acceptable carrier or excipient.

17. A diagnostically useful composition comprising
(a) the composition of claim 12 which is diagnostically or detectably labeled; and
(b) a diagnostically acceptable carrier or excipient.

18. A diagnostically useful composition comprising
(a) the composition of claim 13 which is diagnostically or detectably labeled; and
(b) a diagnostically acceptable carrier or excipient.

19. The diagnostically useful composition of claim 15 wherein the monoclonal antibody, fragment or derivative is labeled with a detectable label selected from the group consisting of a radionuclide, a PET-imageable agent, a MRI-imageable agent, a fluorescer, a fluorogen, a chromophore, a chromogen, a phosphorescer, a chemiluminescer and a bioluminescer.

20. (canceled)

21. The composition of claim 19 wherein the monoclonal antibody, fragment or derivative is labeled with a radionuclide.

22. The composition of claim 21 wherein said radionuclide is one which is detectable in vivo.

23. The composition of claim 22 wherein the radionuclide is detectable by radioimmunoscintigraphy.

24. The composition of claim 21 wherein the radionuclide is selected from the group consisting of ³H, ¹⁴C, ³⁵S, ^{99m}Tc, ¹²³I, ¹²⁵I, ¹³¹I, ¹¹¹In, ⁹⁷Ru, ⁶⁷Ga, ⁶⁸Ga, ⁷²As, ⁸⁹Zr and ²⁰¹Tl.

25. The composition of claim 24 wherein the radionuclide is ¹²⁵I.

26-30. (canceled)

31. The composition of claim 19 wherein the detectable label is a fluorescer or fluorogen.

32. The composition of claim 31 wherein the fluorescer or fluorogen is selected from the group consisting of fluorescein, rhodamine, dansyl, phycoerythrin, phycocyanin, allophycocyanin, o-phthaldehyde, fluorescamine, a fluorescein derivative, Oregon Green, Rhodamine Green, Rhodol Green and Texas Red.

33-34. (canceled)

35. The composition of claim 19 wherein said detectable label is bound to the antibody through one or more diethylenetriaminepentaacetic acid (DTPA) residues that are coupled to the antibody.

36. The composition of claim 35 wherein the detectable label is bound to the antibody through one DTPA residue.

37. The composition of claim 35 useful for MRI diagnosis wherein metal atoms are bound to said DTPA residues.

38. The composition of claim 37 wherein said metal is selected from the group consisting of gadolinium, manganese, copper, iron, gold and europium.

39. The composition of claim 38 wherein said metal is gadolinium.

40-44. (canceled)

45. A therapeutic composition useful for treating a Met-expressing tumor, comprising:
(a) the monoclonal antibody, fragment or derivative of claim 1 in a therapeutically effective amount, and
(b) a pharmaceutically or therapeutically acceptable carrier or excipient.

46. A therapeutic composition useful for treating a Met-expressing tumor, comprising:
(a) the composition of claim 9 in a therapeutically effective amount, and;
(b) a pharmaceutically or therapeutically acceptable carrier or excipient.

47. A therapeutic composition useful for treating a Met-expressing tumor, comprising:
(a) the composition of claim 12 in a therapeutically effective amount, and;
(b) a pharmaceutically or therapeutically acceptable carrier or excipient.

48. (canceled)

49. The therapeutic composition of claim 45 in a form suitable for injection or infusion.

50. The therapeutic composition of claim 45, wherein at least one of the antibodies, fragments or derivatives is bound to, conjugated to, or labeled with a therapeutic moiety.

51. The therapeutic composition of claim 50 wherein the therapeutic moiety is a radionuclide.

52. The therapeutic composition of claim 51 wherein the radionuclide is selected from the group consisting of ⁴⁷Sc, ⁶⁷Cu, ⁹⁰Y, ¹⁰⁹Pd, ¹²⁵I, ¹³¹I, ¹⁸⁶Re, ¹⁹⁹Au, ²¹¹At, ²¹²Pb and ²¹²Bi.

53-57. (canceled)

58. The therapeutic composition of claim 47, wherein at least one of the antibodies, fragments or derivatives is bound to, conjugated to, or labeled with a therapeutic moiety.

59. The therapeutic composition of claim 58 wherein the therapeutic moiety is a radionuclide.

60. The therapeutic composition of claim 59 wherein the radionuclide is selected from the group consisting of ⁴⁷Sc, ⁶⁷Cu, ⁹⁰Y, ¹⁰⁹Pd, ¹²⁵I, ¹³¹I, ¹⁸⁶Re, ¹⁸⁸Re, ¹⁹⁹Au, ²¹¹At, ²¹²Pb and ²¹²Bi.

61-64. (canceled)

65. A kit, comprising:
(a) a labeled first container comprising the antibody, fragment or derivative of claim 1;
(b) a labeled second container comprising a diagnostically or pharmaceutically-acceptable carrier or excipient; and
(c) instructions for using the antibody to diagnose, prognose, monitor or treat a cancerous condition or a tumor in a subject wherein cancer or tumor cells in said subject are known or suspected to express Met,
wherein the antibody, fragment or derivative is effective for diagnosing, prognosing, monitoring or treating said condition and
said labeled container indicates that the antibody can be used for said diagnosing, prognosing, monitoring or treating.

66. A method for detecting the presence of Met (i) on the surface of a cell, (ii) in a tissue, (iii) in an organ or (iv) in a biological sample, which cell, tissue, organ or sample is suspected of expressing Met, comprising the steps of:
(a) contacting the cell, tissue, organ or sample with the composition of claim 15;
(b) detecting the presence of the label associated with the cell, tissue, organ or sample.

67-69. (canceled)

70. The method of claim 66, wherein the contacting and the detecting are in vitro.

71. The method of claim 66 wherein the contacting is in vivo and the detecting is in vitro.

72. The method of claim 66, wherein the contacting and the detecting are in vivo.

73-75. (canceled)

76. The method of claim 72 wherein said detectable label is a radionuclide

77-79. (canceled)

80. The method of claim 76 wherein the radionuclide is selected from the group consisting of ³H, ¹⁴C, ³⁵S, ^{99m}Tc, ¹²³I, ¹²⁵I, ¹³¹I, ¹¹¹In, ⁹⁷Ru, ⁶⁷Ga, ⁶⁸Ga, ⁷²As, ⁸⁹Zr and ²⁰¹Tl.

81-83. (canceled)

84. The method of claim 80 wherein said detecting is by radioimmunoscintigraphy.

85-87. (canceled)

88. The method of claim 84 wherein the radionuclide is ¹²⁵I.

89-91. (canceled)

92 The method of claim 72, wherein the detectable label is an MRI-imageable agent and the detecting is by MRI.

93-95. (canceled)

96. A method for inhibiting (i) the proliferation, migration, or invasion of, Met-expressing tumor cells or (ii) angiogenesis induced by Met-expressing tumor cells, comprising contacting said cells with an effective amount of the therapeutic composition of claim 45.

97-99. (canceled)

100. The method of claim 96 wherein the contacting is in vivo.

101-103. (canceled)

104. The method of claim 100 wherein the therapeutic composition of is in a form suitable for injection or infusion.

105. The method of claim 100 wherein, in the therapeutic composition, at least one of the antibodies, fragments or derivatives is bound to, conjugated to, or labeled with a therapeutic moiety.

106. The method of claim 105 wherein, in the therapeutic composition, the therapeutic moiety is a radionuclide.

107-115. (canceled)

116. A method for treating a subject having a cancerous disease or condition associated with (i) undesired proliferation, migration or invasion of Met-expressing cells or (ii) undesired angiogenesis induced by Met-expressing cells, comprising administering to the subject an effective amount of the therapeutic composition of claim 45.

117-119. (canceled)

120. The method of claim 116 wherein, in the therapeutic composition, at least one of the antibodies, fragments or derivatives is bound to, conjugated to, or labeled with a therapeutic moiety.

121-123. (canceled)

124. The hybridoma cell line deposited in the American Type Culture Collection under Accession Number PTA-4349.

125. The hybridoma cell line deposited in the American Type Culture Collection under Accession Number PTA-4477.
